(19) United States
(12) Patent Application Publication
(10) Pub. No.: US 2024/0271164 A1
(43) Pub. Date: Aug. 15, 2024

(54) SYNTHETIC GENOMIC SAFE HARBORS AND METHODS THEREOF

(71) Applicant: The Regents of the University of California, Oakland, CA (US)

(72) Inventors: Linda L. WALLING, Riverside, CA (US); Peter W. ATKINSON, Riverside, CA (US)

(73) Assignee: The Regents of the University of California, Oakland, CA (US)

(21) Appl. No.: 18/634,406

(22) Filed: Apr. 12, 2024

Related U.S. Application Data
(63) Continuation of application No. PCT/US2023/034566, filed on Oct. 5, 2023.
(60) Provisional application No. 63/413,572, filed on Oct. 5, 2022.

(51) Int. Cl. C12N 15/90; C12N 9/22; C12N 15/11; C12N 15/82

(52) U.S. Cl. CPC C12N 15/907; C12N 9/22; C12N 15/11; C12N 15/8213; C12N 15/902; C12N 2310/20; C12N 2800/80; C12N 2840/203

(57) ABSTRACT

Certain embodiments of the invention provide a synthetic genomic safe harbor in the genome of a cell. Certain embodiments provide a method of creating a synthetic genomic safe harbor in a genome. Certain embodiments of the invention provide a method of genome editing in a cell.

### CROSS REFERENCE TO RELATED APPLICATION

This application claims priority to U.S. Provisional Application No. 63/413,572 filed on 5 Oct. 2022. The entire content of the application referenced above is hereby incorporated by reference herein.

### BACKGROUND OF THE INVENTION

Integration of transgenes into a genome often leads to low levels of gene expression or gene inactivation. Such events limit genetic strategies. Optimal genome sites for expressing transgenes are important in, for example, insect gene-drive control strategies, insect sterile-release control programs, transgenic plants (e.g., designed to express genes for insect control), human cell and gene therapies, and for expression of proteins important for industry, nutrition, and medicine. However, current methods for finding optimal genome sites and for transgene integration have limitations. New strategies are needed.

### SUMMARY OF THE INVENTION

Certain embodiments of the invention provide a synthetic genomic safe harbor (sGSH) as described herein (e.g., a cargo-loaded sGSH comprising a complementation gene and a transgene; or a minimal, receiving sGSH comprising a complementation gene and a landing sequence capable of receiving one or more transgene(s) to be inserted). Certain embodiments of the invention provide a synthetic genomic safe harbor (sGSH) in a genome, the synthetic GSH comprising an exogenous fusion sequence that is inserted at the locus of an endogenous target gene of the genome, wherein the fusion sequence comprises:
(a) a transgene sequence encoding the transgene product, and
(b) a complementation sequence comprising a rescue gene sequence that encodes the target gene product.

Certain embodiments of the invention provide a synthetic genomic safe harbor (sGSH) in a genome, the synthetic GSH comprising an exogenous fusion sequence that is inserted at the locus of an endogenous target gene of the genome, wherein the fusion sequence comprises:
(a) a landing sequence comprising a cutting sequence (e.g., comprising PAM sequence and gRNA sequence), and
(b) a complementation sequence comprising a rescue gene sequence that encodes a target gene product.

Certain embodiments of the invention provide a method of making a synthetic genomic safe harbor (sGSH) as described herein (e.g., a single step method to arrive at a cargo-loaded sGSH directly, or a method of making a receiving sGSH first, and then inserting a transgene into the landing sequence of the receiving sGSH).

In certain embodiments, a synthetic genomic safe harbor described herein is capable of matching the developmental, tissue, and/or cellular expression specificity of a transgene with that of the endogenous target gene or its neighboring gene(s). For example, a synthetic GSH may comprise expression cassettes or promoters capable of matching (temporally and spatially) the developmental, tissue, and/or cellular expression specificity of the transgene with that of the endogenous target gene/the rescued target gene. In certain embodiments, the sGSH comprises two different promoters that are similarly regulated. In certain embodiments, the sGSH comprises two promoters having 100% sequence identity to each other. In certain embodiments, the sGSH comprises one or two promoters having 100% sequence identity to the native promoter sequence of the endogenous target gene.

Certain embodiments of the invention provide a method of making a synthetic GSH in a genome, the method comprising:
inserting an exogenous fusion sequence at the locus of an endogenous target gene of the genome, wherein the insertion of the fusion sequence inactivates the endogenous target gene, wherein the fusion sequence comprises:
(a) a transgene sequence encoding the transgene product, or a landing sequence described herein and
(b) a complementation sequence comprising a rescue gene sequence that encodes the target gene product.

In certain embodiments, the endogenous target gene is not an essential gene (inactivation of which may lead to severe or lethal fitness cost, such as infertility, etc.). In certain embodiments, the endogenous target gene is a non-essential gene (inactivation of which may lead to small or mild fitness cost, such as eye color change, or impaired pair mating etc.). As used herein, an “essential gene” is a gene that inactivation of which (homozygous loss) will result in lethality or stop an individual subject's reproduction and propagation. As used herein, an “non-essential gene” is a gene that inactivation of which (homozygous loss) will not result in lethality or stop an individual subject's reproduction and propagation.

In certain embodiments, the endogenous target gene has a simple structure (e.g., no intron, or only has 1, 2, or 3 short intron(s) of length<1 kb) and a simple regulatory mechanism, e.g., primarily or only regulated by transcriptional control and no alternative splicing.

Certain embodiments of the invention provide a method of delivering a gene of interest (transgene sequence) to a cell comprising a sGSH described herein, comprising inserting a sequence comprising a transgene sequence encoding the transgene product into a landing pad of the sGSH, wherein the sGSH comprises an exogenous fusion sequence comprising: (a) a landing sequence described herein, and (b) a complementation sequence comprising a rescue gene sequence that encodes the target gene product as described herein.

Certain embodiments of the invention provide a polynucleotide as described herein (e.g., comprising an exogenous fusion sequence described herein).

Certain embodiments of the invention provide a method as described herein (e.g., a genome editing method), including a method of delivering a gene of interest to a cell, the method comprising contacting the cell with polynucleotide as described herein.

Certain embodiments of the invention provide a method of genome editing in a cell, comprising inserting an exogenous fusion sequence at the locus of an endogenous target gene, wherein the insertion of the fusion sequence inactivates the endogenous target gene, wherein the fusion sequence comprises (a) a transgene, and (b) a complementation sequence comprising a nucleic acid sequence of the target gene and a promoter sequence for the target gene.

Certain embodiments of the invention provide a method as described herein.

Certain embodiments of the invention provide a nucleic acid sequence described herein (e.g., comprising an exogenous fusion sequence described herein).

Certain embodiments of the invention provide a vector described herein (e.g., comprising an exogenous fusion sequence described herein).

### BRIEF DESCRIPTION OF THE FIGURES

FIG. 1. Genomic Safe Harbors (GSH). The site of transgene insertion impacts level of expression and ability of the transgene to be expressed for many generations. The central genes (the central graph) would be considered an optimal GSH as compared to the left and right graphs (lower expression level) in this schematic drawing.

FIGS. 2A-2C. The power of targets-on-demand (ToD) GSH sites. FIG. 2A. A transgene (light gray) inserts onto a functional target gene (gray) thereby inactivating it, leading to a fitness cost for the whitefly. FIG. 2B. Structure of an exemplary complementation gene, which restores target gene function. This exemplary complementation gene sequence is a fusion of the target gene promoter and the target gene's cDNA (dark gray). FIG. 2C. Integration of the complementation gene and the transgene into the target site occurs. While the target gene itself is inactivated, its gene function is retained due to the expression of the complementation gene.

FIGS. 3A-3C. The ToD complementation scheme. FIG. 3A. A transgene expressing dsRed inserts onto a functional cn gene thereby inactivating it, leading to cn-colored eyes and a fitness cost. FIG. 3B. Structure of an exemplary complementation gene sequence Cn:cn-cDNA that can synthesize the cn RNA and protein. FIG. 3C. Integration of the Cn:cn-cDNA gene and the adjacent dsRed gene using homology directed repair (HDR) and cn homology arms. When integrated into the GWSS cn gene, the native gene is inactive but the Cn:cn-cDNA gene can make a wild-type mRNA and protein. The insects will have wild-type eyes and no fitness cost. The promoter used to drive the transgene could match the developmental, tissue or cellular specificity of the target gene (ToD). FIG. 3C emphasizes that the target gene encodes a single RNA (unique to ToD), is in an open chromatin region and in transcribed region of the genome. As such, this strategy differs from that used by other GSH strategies which avoid insertion into, or near, active genes.

FIG. 4. The unexpected origins of certain high-impact and broad application discoveries in biology and chemistry.

FIG. 5. Research team and expertise.

FIG. 6. Current conventional control strategies are short-lived and have problems. New transgenic strategies are now emerging.

FIG. 7. We focus on creating new genetic methods for the control of hemipteran pests (e.g., using CRISPR-Cas9) to reduce damage to crops.

FIG. 8. In developing tools to create genetic control methods for Glassy-winged sharpshooter (GWSS), there is challenge that is universal to all transgenic technologies. The general challenges to transgenesis relate to the event that when a transgene is inserted into a target locus; the target gene's protein is no longer made and can result in mild to severe fitness costs. In addition, based on the genomic context, the transgene could be expressed at high, medium or low levels or totally silenced. While illustrated with an insect as a model, these challenges exist in all transgenesis experiments.

FIG. 9. Transgenes need an optimal insertion site to function, to provide optimal transgene expression and that no harm is done to the organism.

FIG. 10. Certain reasons for why genomic safe harbors are needed.

FIG. 11. Difficulties and certain reasons for why genomic safe harbors are hard to find.

FIG. 12. Labor- and resources-intensive methods for identifying GSHs. Current methods for identifying GSHs are labor-intensive, time-intensive and expensive. Flow cytometry has been used to identify cells expressing transgenes at high levels in mammals and in one insect (Miyata et al 2022).

FIG. 13. Certain current methods for isolating Genomic Safe Harbors. In plants, large-scale screens with big experimental foot-prints are used. Alternatively, large collections mutants made with fast-neutron have been used to identify putative GSHs. Computational approaches predominate in humans and model organisms that are replete with bioinformatic resources.

FIG. 14. Competitive matrix of approaches to Genomic Safe Harbor Discovery.

FIG. 15. Target-on-demand is a big idea from humble origins.

FIG. 16. The solution of Target-on-demand (ToD) uses rescue genes to create synthetic GSHs.

FIGS. 17A-17C. FIGS. 17A-17C provide non-limiting, exemplary ToD technology. To deploy the ToD technology, three types of genes might be involved. Virtually any gene can be engineered to become a synthetic GSH. In this non-limiting example, we illustrate the ToD concept using GWSS in this figure. A “rescue” gene complements the target gene's function upon transgene cassette insertion and no fitness costs to the organism are incurred. The transgene is expressed at the desired level (high, medium or low depending on the transgenic strategy in the appropriate developmental stage, tissue and cell type. FIG. 17A. To deploy the cargo-loaded ToD technology in a single step, we need three genes. 1) This exemplary cassette has homology arms that allow HDR recombination of the ToD cassette into the target gene locus. A target gene into which we insert our cassette, expressed at a level appropriate for the transgene strategy (e.g., expressed at high levels if high level is proper for the transgene strategy), expressed at the correct developmental stage, in the correct desired tissue and cell type, has neighboring genes which are expressed in a similar manner during development and in tissues and cell types. 2) a rescue gene that expresses the target gene protein. 3) a transgene that confers a value-added trait to the organism. FIG. 17B provides a non-limiting exemplary minimal ToD cassette. This cassette has homology arms that allow HDR recombination of the ToD cassette into the target gene locus. The minimal ToD cassette has the rescue gene that provides the coding region for the rescue gene. In this example, adjacent to the rescue gene is a unique Cas/sgRNA cutting site (with star), which is called the landing pad. FIG. 17C The landing pad can accommodate one or more transgenes. By providing Cas endonuclease, the sgRNA and the donor plasmid, the transgene with homology arms that flank the landing pad sgRNA site is integrated into the synthetic GSH. This affords flexibility to include any transgene gene into the synthetic GSH. FIG. 17B-FIG. 17C. The minimal ToD cassette has a rescue gene and a landing pad, capable of facilitating an exemplary two-step incorporation of transgene. 1) A target gene into which we insert our cassette, expressed at a level appropriate for the transgene strategy, expressed at the correct developmental stage, in the correct desired tissue and cell type, has neighboring genes which are expressed in a similar manner during development and in tissues and cell types. 2) a rescue gene that expresses the target gene protein. 3) a landing pad (box with star) that has a unique sgRNA site to allow transgene insertion. 4) a transgene that is later inserted into the landing pad to confer a value-added trait to the organism.

FIG. 18. An example of how to make a rescue gene (Comparison of native and rescue gene structures). Concepts are illustrated using the cn gene of GWSS. Left graph. Target (putative GSH) gene structure. Target gene promoter, gene including introns and 3′flanking region are shown. The 11 introns of the GWSS gene are not shown. Right graph. The promoter for rescue gene and the rescue gene sequence encoding the product in this example contain the cn promoter and cn cDNA including 5′ and 3′ UTRs. The rescue gene will express the cn protein in the correct cells and tissues at the correct time in development to avoid fitness costs.

FIG. 19. Test the Target- on-demand (ToD) technology with the GWSS cinnabar gene. We use GWSS and the cn target gene and cn rescue gene as an illustration of the ToD technology. GWSS that has the ToD gene cassette integrated into the cn target gene locus will be identified and phenotypes assessed. The transgene could use a promoter with a similar expression program to the target gene to assure correct expression. Alternatively, any other promoter can be used to express the target gene but its level of expression will need to be tested empirically.

FIG. 20. Synthetic genomic safe harbors may accelerate discoveries and deployment of transgenic strategies in major sectors of medicine, biotechnology, agriculture, and insect control. It is the next big idea from humble origins.

FIGS. 21A-21C. The ToD rescue gene complementation scheme. FIG. 21A. A transgene expressing dsRed inserts into a functional cn gene thereby inactivating it, leading to cn-colored eyes and a fitness cost. FIG. 21B. Structure of the cn rescue gene Cn:cn-cDNA that can synthesize the cn RNA and protein. FIG. 21C. Integration of the Cn:cn-cDNA gene and the adjacent dsRed gene using HDR and cn homology arms. When integrated into the GWSS cn gene, the native gene is inactive but the Cn:cn-cDNA gene can make a wild-type mRNA and protein. The insects will have wild-type eyes and no fitness cost.

### DETAILED DESCRIPTION

A major problem in contemporary approaches to gene editing in the medical and agricultural fields relates to the challenges in finding sites into the target organism genome in which cassettes containing beneficial gene(s) can be accurately inserted with no side effects or fitness costs to the individual. Such sites are called genomic safe harbors (GSHs). Certain representative criteria have been proposed in the past to identify GSH computationally, in particular, these putative GSHs should: for example, (1) be >50 kb from a transcriptional start site, (2) not disrupt a transcriptional unit, (3) be >300 kb from miRNAs, and among other considerations. Thus, use of transcriptional unit and coding regions is effectively banned in such computational methods to identify GSHs for inserting a transgene. In model organisms, GSHs have remained difficult or elusive to find due to the immense cost and time needed to construct the genomic resources (e.g., annotated genome, chromosomal level genome assembly, transcriptomes, or knowledge of chromatin accessibility) to perform GSH identification bioinformatically and the absence of cell culture lines (for many organisms) to allow large-scale automated screens.

A simple approach that bypasses these strategies is described herein to create synthetic genomic safe harbors in selected target genes themselves. In this manner, synthetic genomic safe harbor (referred to as synthetic GSH, or sGSH) can be made to allow the insertion of a gene cassette having transgene into virtually any suitable target gene using the target-on-demand (ToD) strategy described herein. Thus, a target gene could be transformed into a synthetic genomic safe harbor. For example, the chosen endogenous target gene could express a single RNA and be surrounded by transcriptionally active genes. These are simple criteria, and the resources are often in place even in non-model organisms. By avoiding costly screening and the need for cell culture platforms or genetically tagged libraries, ToD is a fast and efficient GSH discovery/creation tool that could revolutionize gene-editing and transgenic strategies in all organisms, having especially high impact on non-model organisms and biotechnology.

Thus, the synthetic GSH as described herein comprises 1) a complementation gene (also referred to as a rescue gene); and 2) a transgene, and/or a landing sequence into which a transgene could be inserted. Thus, the synthetic GSH comprises exogenous, recombinant sequence introduced into the edited genome.

In certain embodiments, a synthetic GSH comprises 1) a complementation gene (also referred to as a rescue gene); and 2) a landing sequence. This synthetic GSH does not yet comprise an inserted transgene sequence that encodes a transgene product; such a synthetic GSH is termed a “minimal synthetic GSH” or “receiving synthetic GSH” that is capable of receiving a transgene sequence or for insertion of a transgene sequence.

In other embodiments, a synthetic GSH comprises 1) a complementation gene (also referred to as a rescue gene); and 2) a transgene. Such a synthetic GSH comprising a transgene sequence that encodes a transgene product is termed “cargo-loaded synthetic GSH”.

In certain embodiments, a “receiving synthetic GSH” is introduced into a genome first, and a transgene sequence is then inserted to arrive at a “cargo-loaded synthetic GSH” comprising a transgene sequence.

However, in certain embodiments, introduction of “receiving synthetic GSH” into a genome is not necessary and bypassed, namely, a “cargo-loaded synthetic GSH” comprising an exogenous fusion sequence that comprises a complementation sequence and a transgene sequence may be inserted into the genome directly.

For example, a targeted nuclease such as CRISPR-Cas9 could specifically home to and cut at the genomic locus of an endogenous target gene. A synthetic GSH sequence could be installed into the targeted genomic site via homology directed repair (HDR) or nonhomologous end joining (NHEJ). During this process, the original transcriptional unit of the target gene is disrupted so that functional product would not be expressed from the now disrupted original genomic sequence. However, the successfully installed synthetic GSH at the locus could complement (i.e., rescue) the loss of target gene function. For example, a cargo-loaded synthetic GSH could not only express the transgene but also express the otherwise inactivated target gene, because the synthetic GSH sequence comprises: (a) a transgene sequence encoding the transgene product and (b) a complementation sequence comprising a sequence encoding the target gene product, facilitating expression of the transgene product without fitness cost to host cell thanks to the expression of the rescued target gene product.

As long as the introduced synthetic GSH in the edited genome is capable of facilitating expression of the transgene product, and rescue gene product (which is identical to the endogenous target gene product), the fitness cost from inserting the synthetic GSH into the target gene locus could be minimized or prevented. A variety of synthetic GSH embodiments capable of achieving such functional outcome are described herein.

Briefly, the cargo-loaded synthetic GSH comprises at least two genes (transgene gene sequence and rescue gene sequence) sequences that encode two products (transgene product and target gene product). In its simplest form of execution (smaller synthetic GSH construct), the rescue gene could be placed upstream of the transgene. Alternatively, the transgene could be placed upstream of the rescue gene, which may require delivery of the entire target gene promoter and cDNA. The two products could be two separate and distinct products, or the two products may be a target gene-transgene fusion protein. It is to be understood that in certain embodiments, the target gene's promoter is not proposed to drive the transgene so the two genes should be expressed under two separate promoters respectively; however, it is also possible to express two genes under a single promoter using an IRES (internal ribosomal entry site) sequence, or 2A peptide (e.g., T2A) encoding sequence in between the two genes sequences that encode the products (e.g., two small gene products and/or to save from using a second promoter of great length).

Accordingly, certain embodiments of the invention provide a synthetic genomic safe harbor (GSH) in a genome, and a method of making a synthetic genomic safe harbor in a genome. In certain embodiments, the genome is a mammalian genome, a plant genome, an insect genome, a fungal genome, an oomycete genome, or a bacterium genome

In certain embodiments, the insect genome is from an insect Bemisia tabaci or Homalodisca vitripennis, but the technology can be applied to any insect species amenable to gene editing.

In certain embodiments, the insect genome is a genome of an insect in the order Diptera, Lepidoptera, Coleoptera, Hemiptera, or Orthoptera. In certain embodiments, the insect genome is a genome of an insect in the Aleyrodidae family. In certain embodiments, the insect genome is a genome of a psyllid, sharpshooter, leafhopper, planthopper, aphid, Bagruda bug, Lygus bug, box elder bug, chili thrip, crape myrtle bark scale, four-lined plant bug, pink hibiscus mealybug, scale insect, cycad aulacaspis scales, or wax scales on holly.

### Synthetic Genomic Safe Harbor (GSH)

Certain embodiments of the invention provide a synthetic genomic safe harbor (GSH) in a genome, the synthetic GSH comprising an exogenous fusion sequence that is inserted at the locus of an endogenous target gene, wherein the exogenous fusion sequence comprises:
(a) a transgene sequence encoding the transgene product, and/or a landing sequence, and
(b) a complementation sequence comprising a sequence (i.e., a rescue gene sequence) that encodes the target gene product

Certain embodiments of the invention provide a synthetic genomic safe harbor (e.g., a cargo-loaded sGSH) in a genome, the synthetic GSH comprising an exogenous fusion sequence that is inserted at the locus of an endogenous target gene, wherein the exogenous fusion sequence comprises:
(a) a transgene sequence encoding the transgene product, and
(b) a complementation sequence comprising a sequence (i.e., a rescue gene sequence) that encodes the target gene product.

Certain embodiments of the invention provide a synthetic genomic safe harbor (e.g., a receiving sGSH) in a genome, the synthetic GSH comprising an exogenous fusion sequence that is inserted at the locus of an endogenous target gene of the genome, wherein the fusion sequence comprises:
(a) a landing sequence comprising a cutting sequence, and
(b) a complementation sequence comprising a rescue gene sequence that encodes a target gene product.

The term “cutting sequence” refers to a nucleic acid sequence capable of being cut by a targeted nuclease, such as a Cas nuclease, and the nucleic acid sequence is not naturally present at the locus of the endogenous target gene. In certain embodiments, the cutting sequence is not naturally present throughout the entire original genomic sequence of the genome (e.g., no off-target effect when the cutting sequence is cut by a targeted nuclease). In certain embodiments, the cutting sequence is a unique sequence, wherein the locus of the endogenous target gene or the entire original genomic sequence of the genome has no sequence having 100% sequence identity to the cutting sequence. In certain embodiments, the locus of the endogenous target gene or the original genomic sequence of the genome has no sequence having at least 99% (e.g., at least 97%, 95%, 90%, 85%, 80%, or 75%) sequence identity to the cutting sequence. In certain embodiments, the locus of the endogenous target gene or the original genomic sequence of the genome has no sequence having at least 70% (e.g., at least 65%, 60%, 55%, or 50%) sequence identity to the cutting sequence.

In certain embodiments, the cutting sequence comprises a protospacer adjacent motif (PAM) site sequence, and a gRNA related sequence (so that a Cas nuclease could cut the cutting sequence).

In certain embodiments, the cutting sequence comprises a PAM sequence, and a gRNA related sequence, wherein the gRNA related sequence has a length of about 18-25 nt, 19-23 nt, or 20-22 nt (e.g., about 20 nt). In certain embodiments, the gRNA related sequence's first 6-7 nt adjacent to the PAM sequence is a unique sequence, the locus of the endogenous target gene or the entire original genomic sequence of the genome has no sequence having 100% sequence identity to it.

In certain embodiments, the gRNA related sequence is a unique sequence, wherein the locus of the endogenous target gene or the entire original genomic sequence of the genome has no sequence having 100% sequence identity to the gRNA related sequence. In certain embodiments, the locus of the endogenous target gene or the original genomic sequence of the genome has no sequence having at least 99% (e.g., at least 97%, 95%, 90%, 85%, 80%, or 75%) sequence identity to the gRNA related sequence. In certain embodiments, the locus of the endogenous target gene or the original genomic sequence of the genome has no sequence having at least 70% (e.g., at least 65%, 60%, 55%, or 50%) sequence identity to the gRNA-related sequence.

In certain embodiments, the cutting sequence (e.g., comprising PAM site sequence and gRNA-related sequence) has a length of about 19-32 nt. In certain embodiments, the cutting sequence has a length of about 20-29 nt. In certain embodiments, the cutting sequence has a length of about 20-28nt. In certain embodiments, the cutting sequence has a length of about 20-26 nt. In certain embodiments, the cutting sequence has a length of about 20-24 nt.

In certain embodiments, the cutting sequence has a GC content of about 40-60%. In certain embodiments, the cutting sequence has a GC content of about 45-55%. In certain embodiments, the cutting sequence has a GC content of about 50%.

In certain embodiments, the landing sequence comprises two or more unique cutting sequences (e.g., each unique cutting sequence is separated by at least about 100 bp filler sequence). The nature of the filler sequence is not important so long as the filler sequence is different from all unique cutting sequences that the filler sequence will not be cut by a targeted nuclease that cut at a cutting sequence. In certain embodiments, the filler sequence has a length of about 100-500 nt, 100-400 nt, 100-300 nt, or 100-250 nt. In certain embodiments, the filler sequence is not homologous to sequence at the locus of the endogenous target gene. In certain embodiments, the filler sequence is homologous to sequence at the locus of the endogenous target gene.

In certain embodiments, the landing sequence comprises one cutting sequence and one or two filler sequence(s) that separate the cutting sequence from other sequences on the exogenous fusion sequence (e.g., such as the rescue gene sequence, certain regulatory sequences, and/or homology arm sequence).

In certain embodiments, the landing sequence has a length of about 200-600 nt. In certain embodiments, the landing sequence has a length of about 300-550 nt. In certain embodiments, the landing sequence has a length of about 400-500 nt.

As used herein, the term “landing sequence” or “landing pad” refers to a nucleic acid sequence wherein a transgene sequence could be inserted into, and the nucleic acid sequence is not naturally present at the locus of the endogenous target gene. In certain embodiments, the landing sequence is not naturally present throughout the entire original genomic sequence of the genome. In certain embodiments, the landing sequence is a unique sequence, wherein the locus of the endogenous target gene or the entire original genomic sequence of the genome has no sequence having 100% sequence identity to the landing sequence. In certain embodiments, the locus of the endogenous target gene or the original genomic sequence of the genome has no sequence having at least 99% (e.g., at least 97%, 95%, 90%, 85%, 80%, or 75%) sequence identity to the landing sequence. In certain embodiments, the locus of the endogenous target gene or the original genomic sequence of the genome has no sequence having at least 70% (e.g., at least 65%, 60%, 55%, or 50%) sequence identity to the landing sequence. In certain embodiments, the landing sequence comprises one cutting sequence. In certain embodiments, the landing sequence comprises one or more (e.g., two or more) cutting sequences, and one or more filler sequences.

As used herein, the term “the locus of an endogenous target gene” refers to the genomic locus of the single expression cassette of regulatory sequences and encoding sequence for the endogenous target gene (no other gene product, or expression cassette of other gene product is included in this specific locus of the endogenous target gene). However, this specific locus of the endogenous target gene could be located in a genomic region with actively transcribed, neighboring gene(s). As used herein, the term “encoding sequence”, “sequence that encodes a product”, or “sequence encoding a product” refers to the encoding nucleic acid sequence, such as exon(s) sequences (e.g., cDNA), or exon(s) and intron(s) sequence that could be transcribed and processed into an RNA (e.g., mRNA). In certain embodiments, the encoding sequence is a full-length encoding sequence that encodes the entire product, for example, a full-length cDNA sequence that encodes the entire product.

In certain embodiments, the rescue gene sequence (e.g., full-length cDNA sequence) encodes the entire target gene product.

In certain embodiments, the rescue gene sequence comprises partial cDNA sequence fused to exon(s)/intron(s) sequence for the endogenous target gene (e.g., partial downstream cDNA sequence is fused to upstream exon(s)/intron(s)), wherein the rescue gene sequence encodes the entire target gene product.

In certain embodiments, the rescue gene sequence comprises full-length cDNA that comprises native encoding sequence of the endogenous target gene (i.e., a full-length cDNA having 100% sequence identity to the native exon sequence(s) of the endogenous target gene).

In certain embodiments, the rescue gene sequence comprises full-length cDNA that does not comprise an altered codon(s) relative to the native encoding sequence (such as exon sequence(s), or in mRNA) of the endogenous target gene.

In certain embodiments, the rescue gene sequence comprises full-length cDNA sequence having at least 98%, 99%, or 100% sequence identity to the native encoding sequence of the target gene.

In certain embodiments, the complementation sequence further comprises a promoter sequence for the target gene (i.e., the rescue gene), therefore, the complementation sequence may comprise the rescue gene sequence encoding the target gene product, and a promoter sequence for the rescue gene. In certain embodiments, the complementation sequence further comprises 5′UTR sequence and/or 3′ UTR sequence.

In certain embodiments, the cDNA could be recoded to minimize nucleic acid sequence identity with the endogenous target gene. In these cases, the protein derived from the recoded cDNA region is identical to the endogenous target gene protein.

In certain embodiments, the rescue gene sequence has a length that is shorter than the native sequence of the endogenous target gene (e.g., the rescue gene sequence lacking one or more, or all intron sequences of the endogenous target gene). In certain embodiments, the rescue gene sequence comprises one or more introns of the endogenous target gene but not all intron sequences of the endogenous target gene. In certain embodiments, the rescue gene sequence is missing at least one intron of the endogenous target gene. In certain embodiments, the rescue gene sequence does not comprise intron(s) of the endogenous target gene. In certain embodiments, the rescue gene sequence comprises the cDNA sequence of the endogenous target gene.

In certain embodiments, the rescue gene sequence has a length that is the same as the length of the native sequence of the endogenous target gene (e.g., preserving all intron sequences of the endogenous target gene).

In certain embodiments, the endogenous target gene does not comprise intron(s). In these cases, the rescue gene sequence has the same length as that of the endogenous target gene. In these cases, alternative regulatory sequence (e.g., 3′UTRs) and/or use of alternate codons may be used to minimize gene encoding sequence identity between the endogenous target gene and the rescue gene.

### Promoter(s)

In certain embodiments, the exogenous fusion sequence comprises a promoter sequence.

In certain embodiments, the exogenous fusion sequence comprises a promoter for the target gene (i.e., the rescue gene). In certain embodiments, the exogenous fusion sequence further comprises a promoter sequence for the transgene.

Thus, in certain embodiments, the exogenous fusion sequence comprises a first promoter sequence and a second promoter sequence. In certain embodiments, the two separate promoter sequences comprise different nucleic acid sequences. In certain embodiments, the two separate promoter sequences both comprise the same nucleic acid sequence.

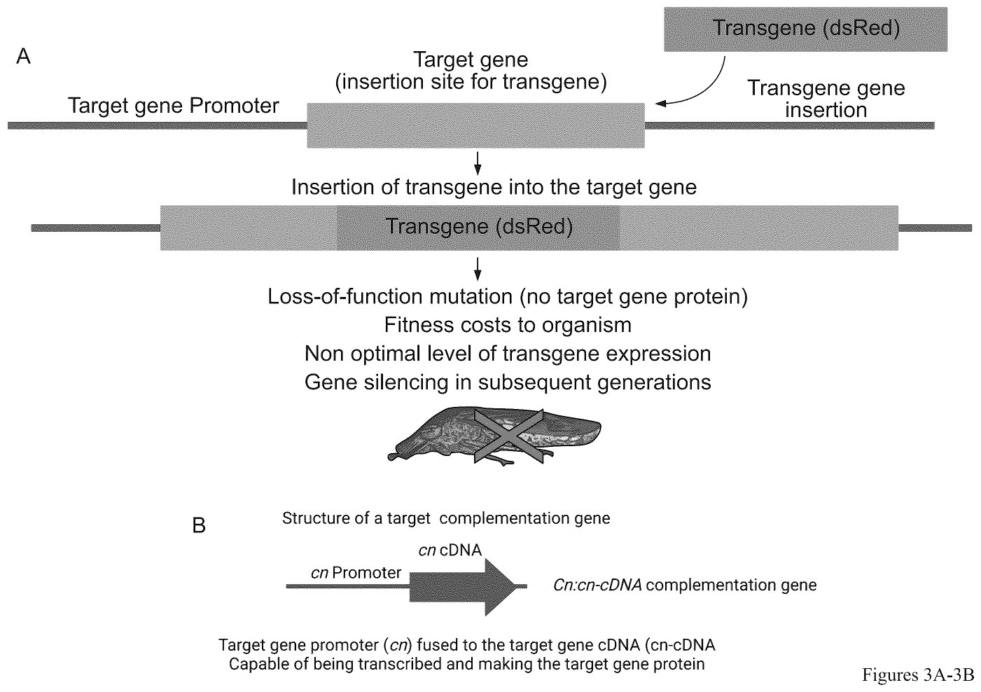

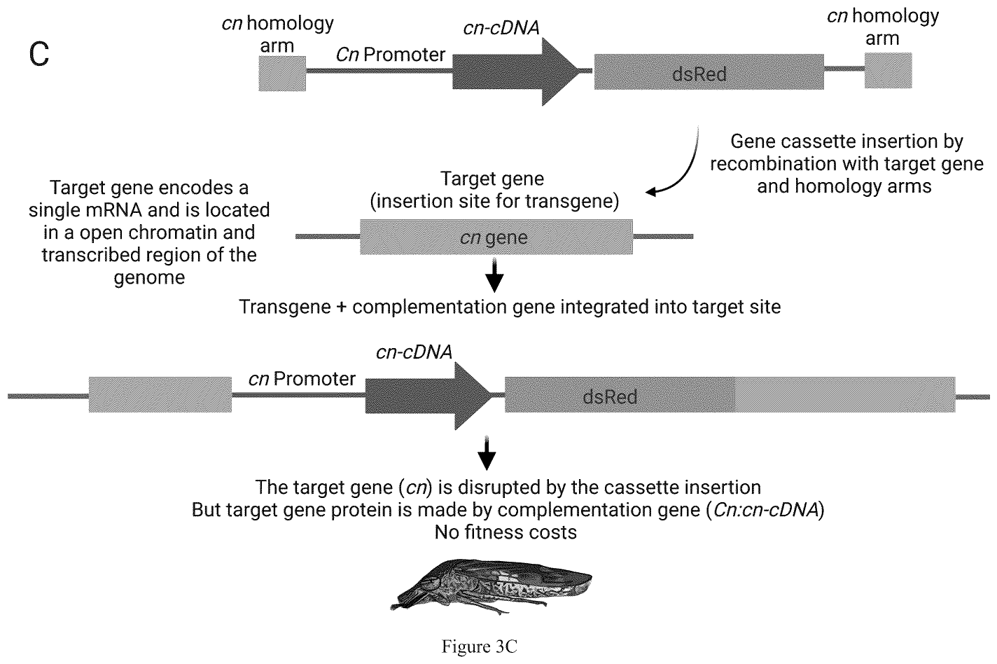

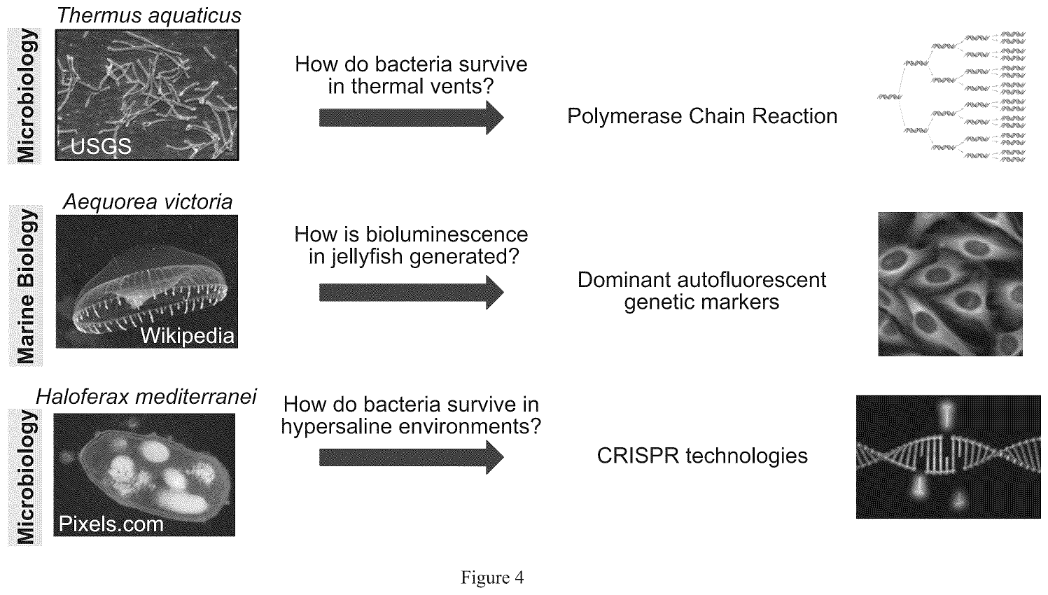

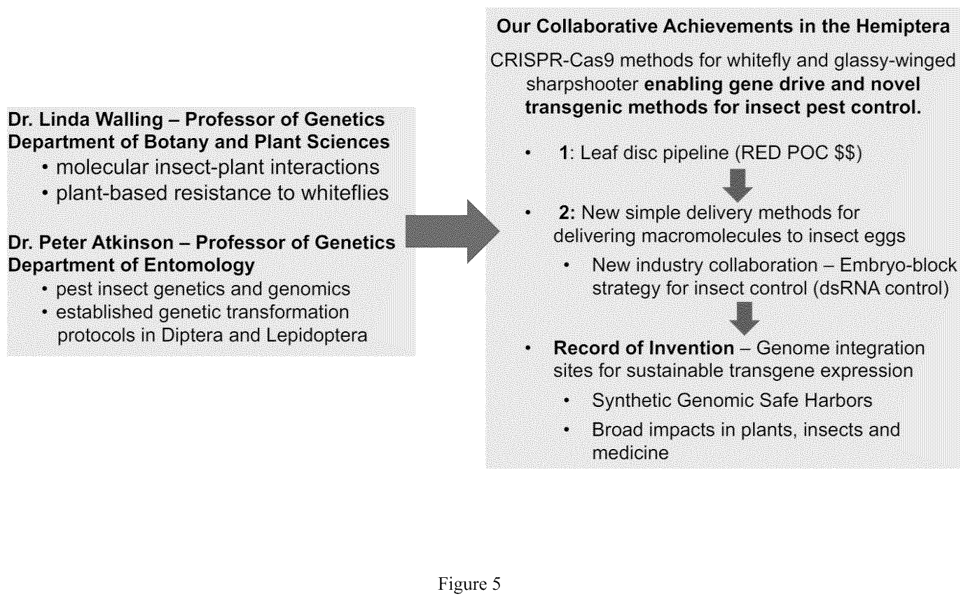

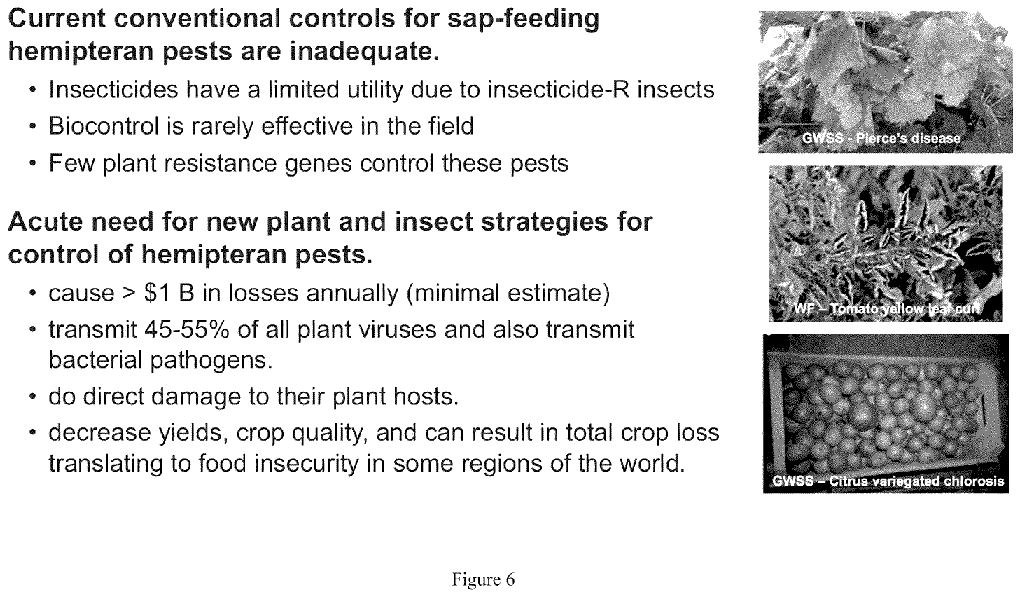

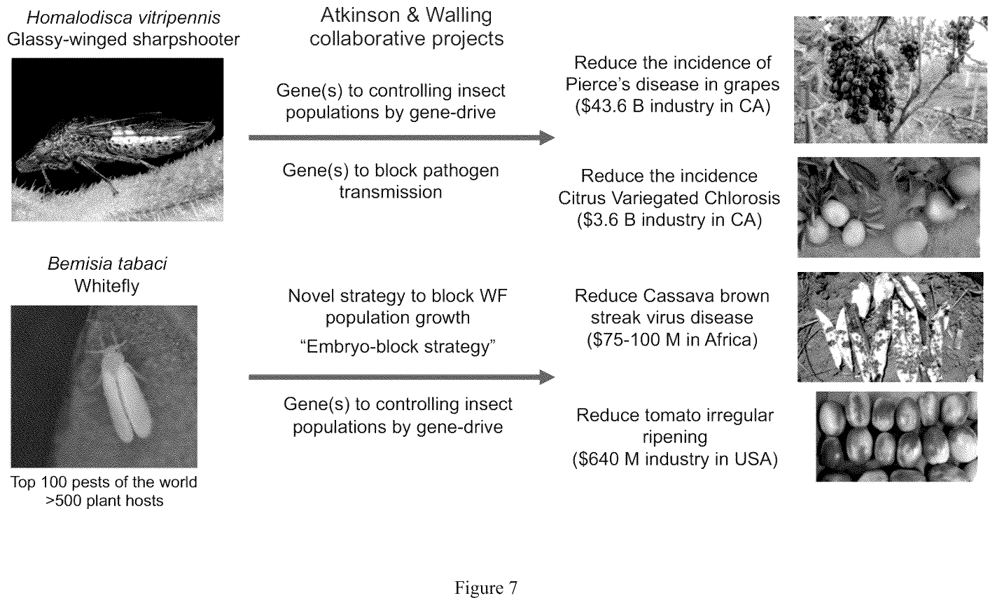

In certain embodiments, the promoter sequence for the rescue gene comprises the native promoter sequence for the endogenous target gene. For example, as shown in FIG. 3C, the promoter sequence for the target gene cn (i.e., rescue gene cn) comprises the native cn promoter nucleic acid sequence.

In certain embodiments, the promoter sequence for the rescue gene comprises a non-native promoter sequence for the target gene. In certain embodiments, the non-native promoter comprises a viral promoter sequence. In certain embodiments, the non-native promoter is a viral promoter suitable for insects (e.g., a baculovirus promoter such as OpIE2). In certain embodiments, the non-native promoter is a viral promoter suitable for mammalian cells (e.g., a CMV promoter). In certain embodiments, the non-native promoter is a viral promoter suitable for plants (e.g., a cauliflower mosaic virus (CaMV) promoter such as CaMV35S). In certain embodiments, the non-native promoter is a promoter suitable for bacteria. In certain embodiments, the non-native promoter is a bacteriophage promoter (e.g., a T7 promoter). In certain embodiments, the promoter for the transgene is a promoter suitable for fungi or oomycete. In certain embodiments, the non-native promoter is a non-viral promoter (e.g., a protomer derived from a mammalian genome, a plant genome, an insect genome, a fungal genome, an oomycete genome, or a bacteria genome).

In certain embodiments, the promoter for the rescue gene is a constitutive promoter In certain embodiments, the promoter for the rescue gene is an inducible promoter. In certain embodiments, the promoter for the rescue gene is a tissue-specific promoter. In certain embodiments, the exogenous fusion sequence (that comprises or does not comprise a landing sequence) further comprises a promoter sequence for the transgene.

In certain embodiments, the exogenous fusion sequence further comprises an optional promoter sequence (e.g., that is downstream of the complementation sequence, and upstream of the landing sequence). Such optional promoter sequence might be suitable for driving expression of a transgene encoding sequence once the transgene encoding sequence is inserted into the landing sequence.

In certain embodiments, the promoter for the transgene is a constitutive promoter. In certain embodiments, the promoter for the transgene is an inducible promoter. In certain embodiments, the promoter for the transgene is a tissue-specific promoter.

In certain embodiments, the promoter sequence for the transgene comprises a viral promoter sequence. In certain embodiments, the promoter for the transgene is a viral promoter suitable for insects (e.g., a baculovirus promoter such as OpIE2). In certain embodiments, the promoter for the transgene is a viral promoter suitable for mammalian cells (e.g., a CMV promoter). In certain embodiments, the promoter for the transgene is a viral promoter suitable for plants (e g., a cauliflower mosaic virus (CaMV) promoter such as CaMV35S) In certain embodiments, the promoter for the transgene is a promoter suitable for bacteria. In certain embodiments, the promoter for the transgene is a bacteriophage promoter (e.g., a T7 promoter). In certain embodiments, the promoter for the transgene is a promoter suitable for fungi or oomycete. In certain embodiments, the promoter for the transgene is a non-viral promoter (e.g., a protomer derived from a mammalian genome, a plant genome, an insect genome, a fungi genome, an oomycete genome, or a bacteria genome).

In certain embodiments, the exogenous fusion sequence comprises one promoter sequence. In certain embodiments, the exogenous fusion sequence could drive transcription of an RNA and co-expression of both rescue gene product and transgene product from the RNA. In certain embodiments, the fusion sequence comprises an internal ribosomal entry site (IRES) sequence, or a 2A peptide (also referred to as 2A self-cleaving peptide, e.g., T2A, P2A, E2A, or F2A) encoding sequence placed between the complementation sequence and the transgene sequence. For example, in certain embodiments, rescue gene (upstream) and transgene (downstream) could be expressed under one promoter for the rescue gene, and the transgene sequence does not have its own separate promoter sequence. In certain embodiments, transgene (upstream) and rescue gene (downstream) could be expressed under one promoter for the transgene, and the rescue gene sequence does not have its own separate promoter sequence. Thus, in certain embodiments, the exogenous fusion sequence comprises one expression cassette comprising one promoter, and an IRES sequence or 2A peptide encoding sequence between two genes sequences. In certain embodiments, exogenous fusion sequence comprises 3′-regulatory sequence (e.g., 3′-UTR sequence) in the expression cassette. In certain embodiments, exogenous fusion sequence comprises 5′-regulatory sequence and/or 3′-regulatory sequence in the expression cassette. In certain embodiments, exogenous fusion sequence comprises 5′-UTR sequence and/or 3′-UTR sequence in the expression cassette In certain embodiments, the exogenous fusion sequence comprises two expression cassettes (two separate promoters for each of the two genes respectively, thus, one expression cassette for rescue gene product and another expression cassette for transgene product). In certain embodiments, exogenous fusion sequence further comprises 3′-regulatory sequence (e.g., 3′-UTR sequence) in each expression cassette. In certain embodiments, exogenous fusion sequence comprises 5′-regulatory sequence and/or 3′-regulatory sequence in each expression cassette. In certain embodiments, exogenous fusion sequence comprises (i) 5′-UTR sequence and/or 3′-UTR sequence in a first expression cassette (e.g., for rescue gene or for transgene), and (ii) 5′-UTR sequence and/or 3′-UTR sequence in a second expression cassette (e.g., for transgene or for rescue gene).

In certain embodiments, the exogenous fusion sequence comprises a first expression cassette capable of expressing rescue gene product (i.e., target gene product), and a second expression cassette capable of expressing transgene product.

In certain embodiments, the exogenous fusion sequence comprises a first expression cassette capable of expressing rescue gene product (i.e., target gene product), a second expression cassette capable of expressing a first transgene product and a third expression cassette capable of expressing a second transgene product.

In certain embodiments, the exogenous fusion sequence comprises a complementation sequence as described herein, a first transgene sequence encoding a first transgene product (e.g., Cas nuclease, or gRNA), and a second transgene sequence encoding a second transgene product (e.g., gRNA, or Cas nuclease). In certain embodiments, a transgene product is an sgRNA gene (U6:sgRNA), or a Cas9, or Cas9-t2A-dsRed gene, or another value added transgene such as one that encodes an enzyme for production of the chemical or protein of interest). Any of these could be added via an sgRNA specific for the landing pad site (landing sequence) adjacent to the rescue gene.

In certain embodiments, the exogenous fusion sequence comprises only one transgene sequence.

In certain embodiments, the exogenous fusion sequence does not comprise a transgene sequence that encodes a fluorescent protein product. In certain embodiments, the exogenous fusion sequence does not comprise a transgene sequence that encodes a gRNA product. In certain embodiments, the exogenous fusion sequence does not comprise a transgene sequence that encodes a Cas nuclease product. In certain embodiments, the exogenous fusion sequence does not comprise a transgene sequence that encodes a product selected from the group consisting of a fluorescent protein, a Cas nuclease, and a gRNA.

In certain embodiments, the exogenous fusion sequence comprises a promoter sequence capable of driving expression in a germline cell (e.g., an insect germline cell). In certain embodiments, the exogenous fusion sequence comprises a first promoter sequence and a second promoter sequence, both of which are capable of driving expression in a germline cell (e g., an insect germline cell).

In certain embodiments, the insect cell is from Bemisia tabaci and Homalodisca vitripennis, but the technology can be applied to any insect species amenable to gene editing. In certain embodiments, the insect cell is from an insect in the order Diptera, Lepidoptera, Coleoptera, Hemiptera, or Orthoptera. In certain embodiments, the insect cell is from an insect in the Aleyrodidae family. In certain embodiments, the insect cell is a cell of psyllid, sharpshooter, leafhopper, planthopper, aphid, Bagruda bug, Lygus bug, box elder bug, chili thrip, crape myrtle bark scale, four-lined plant bug, pink hibiscus mealybug, scale insect, cycad aulacaspis scales, or wax scales on holly. In certain embodiments, the insect cell is not a mosquito cell.

### Certain Exemplary Fusion Sequence Design Embodiments

In certain embodiments, the exogenous fusion sequence, from 5′ to 3′, comprises a) the complementation sequence and b) the landing sequence, or the transgene sequence (i.e., the landing sequence or the transgene sequence is downstream of the complementation sequence). For example, in certain embodiments, the exogenous fusion sequence, from 5′ to 3′, comprises:
1) the complementation sequence (e.g., comprising a full promoter sequence for the rescue gene, and a sequence encoding the target gene product), and
2) the landing sequence, or the transgene sequence.

In certain embodiments, the exogenous fusion sequence, from 5′ to 3′, comprises:
1) the complementation sequence (e.g., comprising a full promoter sequence for the rescue gene, and a sequence encoding the target gene product),
2) a promoter sequence for the transgene, and
3) the landing sequence, or the transgene sequence.

In certain embodiments, the exogenous fusion sequence, from 5′ to 3′, comprises:
1) the complementation sequence (e.g., comprising a full promoter sequence for the rescue gene, and a sequence encoding the target gene product),
2) an IRES sequence or 2A peptide encoding sequence, and
3) the landing sequence, or the transgene sequence.

In certain embodiments, the exogenous fusion sequence, from 5′ to 3′, comprises a) the landing sequence, or the transgene sequence, and b) the complementation sequence (i.e., the landing sequence, or the transgene sequence is upstream of the complementation sequence). For example, in certain embodiments, the exogenous fusion sequence, from 5′ to 3′, comprises:
1) the landing sequence, or the transgene sequence, and
2) the complementation sequence (e.g., comprising a full promoter sequence for the rescue gene, and a sequence encoding the target gene product).

In certain embodiments, the exogenous fusion sequence, from 5′ to 3′, comprises:
1) a promoter sequence for the transgene,
2) the landing sequence, or the transgene sequence, and
3) the complementation sequence (e.g., comprising a full promoter sequence for the rescue gene and a sequence encoding the target gene product).

In certain embodiments, the exogenous fusion sequence, from 5′ to 3′, comprises:
1) a promoter sequence for the transgene,
2) the landing sequence, or the transgene sequence,
3) an IRES sequence, or 2A peptide encoding sequence, and
4) the complementation sequence (e.g., comprising a full sequence encoding the target gene product).

In certain embodiments, the complementation sequence comprises a promoter sequence for the rescue gene, wherein the promoter sequence is homologous to, or is the native promoter sequence for the endogenous target gene. Accordingly, for example, if a targeted nuclease cuts the original genome near or at the junction between native promoter sequence and encoding sequence of the endogenous target gene, the promoter sequence comprised within the exogenous fusion sequence could serve as upstream homology arm to facilitate integration. Therefore, the exogenous fusion sequence may already comprise a homologous sequence (e.g., promoter sequence (or a portion thereof) as upstream homology arm, or as a non-limiting example, a promoter sequence (or a portion thereof) and exon sequence (or a portion thereof) could together serve as upstream homology arm) in the complementation sequence.

### Additional Flanking Sequence(s)

In certain embodiments, the exogenous fusion sequence further comprises one or two flanking sequence that is homologous to sequence at the locus of the endogenous target gene. In certain embodiments, the one or two flanking sequence is at least 95%, 96%, 97%, 98%, 99%, or 100% homologous to sequence at the locus of the endogenous target gene described herein. In certain embodiments, the exogenous fusion sequence further comprises only one flanking sequence (e.g., the exogenous fusion sequence only comprises one 3′ downstream flanking homology arm sequence and does not comprise any upstream flanking sequence because the complementation sequence of the exogenous fusion sequence already has a promoter sequence that could serve as upstream homology arm).

In certain embodiments, the exogenous fusion sequence, from 5′ to 3′, comprises:
1) the complementation sequence (e.g., comprising a sequence encoding the target gene product, and a promoter sequence for the rescue gene),
2) the landing sequence, or the transgene sequence, and
3) a flanking sequence (i.e., downstream flanking homology arm sequence).

In certain embodiments, the 3′ flanking sequence is homologous to the encoding sequence and/or 3′ regulatory sequence at the locus of the endogenous target gene on the unedited genome. In certain embodiments, the 3′-flanking sequence is about 500 to 1000 nt in length. In certain embodiments, the 3′-flanking sequence is homologous to a downstream region of the endogenous target gene. In certain embodiments, the 3′-flanking sequence is homologous to the last exon. In certain embodiments, the 3′-flanking sequence is homologous to sequence downstream of the last exon. In certain embodiments, the 3′-flanking sequence is homologous to the 3′-regulatory sequence of the endogenous target gene.

In certain embodiments, the 3′-flanking sequence is homologous to exon 1, intron 1, or exon 1 and intron 1 of the endogenous target gene.

In certain embodiments, the exogenous fusion sequence may comprise two flanking sequences (e.g., see FIG. 3C).

Accordingly, in certain embodiments, the exogenous fusion sequence further comprises one or two flanking sequences that are homologous to sequences at the locus of the endogenous target gene. In certain embodiments, each flanking sequence independently has a length of about 100, 150, 200, 250, 300, 400, 500, 600, 700, 800, 900, 1000, 1100, 1200, 1300, 1400, 1500, 1600, 1700, 1800, 1900, or 2000 nt. In certain embodiments, one or both flanking sequences independently have a length of about 100-2000 nt, 100-350 nt, 100-300 nt, 100-200 nt, 300-1200 nt, 500-1600 nt, 500-1000 nt, or 100-2000 nt. In certain embodiments, one or both flanking sequences have a length of about 100-1500 nt, 500-1000 nt, or 600-1000 nt. In certain embodiments, one or both flanking sequences have a length of about 500 nt or 1000 nt.

In certain embodiments, one or both flanking sequences are homologous to a segment of the target gene sequence. As a non-limiting example for illustration purpose, if an exemplary endogenous target gene comprises exon 1, intron 1, exon 2, and the target gene is cut by a targeted nuclease in the middle of intron 1, to facilitate integration, the exogenous fusion sequence of the synthetic GSH may comprise a first flanking sequence that is homologous to the upstream segment of the severed intron 1, and a second flanking sequence that is homologous to the downstream segment of the severed intron 1.

In certain embodiments, the first flanking sequence is homologous to a sequence that is 800-1000 nt upstream of the cut site, and the second flanking sequence is homologous to a sequence that is 800-1000 nt downstream of the cut site.

As another non-limiting example for illustration purpose, if a targeted nuclease cuts the target genome at the junction between regulatory sequence (e.g., promoter sequence or 5′ untranslated region sequence) and exon 1, to facilitate integration, the exogenous fusion sequence of the synthetic GSH may comprise a first flanking sequence (i.e., upstream flanking homology arm) that is homologous to the regulatory sequence (e.g., promoter sequence, and/or 5′-untranslated region sequence), and a second flanking sequence (i.e., downstream flanking homology arm) that is homologous to exon 1 sequence.

As another non-limiting example for illustration purpose, if a targeted nuclease cuts the target genome at the regulatory sequence (e.g., promoter sequence or 5′ untranslated region sequence), to facilitate integration, the fusion sequence of the synthetic GSH may comprise a first flanking sequence that is homologous to upstream segment of the severed regulatory sequence (e.g., promoter sequence, or 5′ untranslated region sequence), and a second flanking sequence that is homologous to the downstream segment of the severed regulatory sequence (e.g., promoter sequence, or 5′ untranslated region sequence).

In certain embodiments, the exogenous fusion sequence, from 5′ to 3′, comprises:
1) a first flanking sequence,
2) the complementation sequence (e.g., comprising a sequence encoding the target gene product, and a promoter sequence for the rescue gene),
3) the landing sequence, or the transgene sequence, and
4) a second flanking sequence.

In certain embodiments, the exogenous fusion sequence, from 5′ to 3′, comprises:
1) a first flanking sequence,
2) the complementation sequence (e.g., comprising a sequence encoding the target gene product, and a promoter sequence for the rescue gene),
3) a promoter sequence for the transgene,
4) the landing sequence, or the transgene sequence, and
5) a second flanking sequence.

In certain embodiments, the exogenous fusion sequence, from 5′ to 3′, comprises:
1) a first flanking sequence,
2) the complementation sequence (e e.g., comprising a sequence encoding the target gene product, and a promoter sequence for the rescue gene),
3) an IRES sequence, or 2A peptide encoding sequence,
4) the landing sequence, or the transgene sequence, and
5) a second flanking sequence.

In certain embodiments, the exogenous fusion sequence, from 5′ to 3′, comprises the transgene sequence and the complementation sequence (i.e., the transgene sequence is upstream of the complementation sequence). For example, in certain embodiments, the exogenous fusion sequence, from 5′ to 3′, comprises:
1) a first flanking sequence,
2) the landing sequence, or the transgene sequence,
3) the complementation sequence (e.g., comprising a sequence encoding the target gene product, and a promoter sequence for the rescue gene), and
4) a second flanking sequence.

In certain embodiments, the exogenous fusion sequence, from 5′ to 3′, comprises:
1) a first flanking sequence,
2) a promoter sequence for the transgene,
3) the landing sequence, or the transgene sequence,
4) the complementation sequence (e.g., comprising a sequence encoding the target gene product, and a promoter sequence for the rescue gene), and
5) a second flanking sequence.

In certain embodiments, the exogenous fusion sequence, from 5′ to 3′, comprises:
1) a first flanking sequence,
2) a promoter sequence for the transgene,
3) the landing sequence, or the transgene sequence,
4) an IRES sequence or 2A peptide encoding sequence,
5) the complementation sequence (e.g., comprising a sequence encoding the target gene product), and
6) a second flanking sequence.

As used herein, the term “original genomic sequence” or “native genomic sequence” refers to the untouched genomic sequence that is not edited or engineered by insertion of a synthetic GSH as described herein.

As used herein, the term “target gene” refers to an endogenous target gene in a genome that is suitable for insertion of a synthetic GSH as described herein. For example, in certain embodiments, the target gene encodes a protein. In certain embodiments, the target gene encodes an RNA that does not have alternatively spliced RNA isoforms. For example, in certain embodiments, the target gene encodes a single protein that does not have other isoforms derived from alternative splicing events. In certain embodiments, the target gene is in a transcriptionally active region of the genome. In certain embodiments, the target gene is located at a DNase I hypersensitive site (DHS) and/or open chromatin such as unmethylated region of the genome. In certain embodiments, the target gene is in a transcriptionally active region that contains two or more genes, for example, the target gene and its adjacent gene(s) are all in a transcriptionally active status. In certain embodiments, the target gene is a single-copy gene in the genome. In certain embodiments, the target gene encodes a non-coding RNA (e.g., miRNA or lncRNA). In certain embodiments, the target gene encodes a microRNA (miRNA). In certain embodiments, the target gene encodes a long non-coding RNA (lncRNA).

In certain embodiments, the synthetic GSH described herein is located within a cluster of genes on the genome. For example, in certain embodiments, the synthetic GSH may be inserted at the locus of one endogenous target gene without disrupting neighboring gene(s). In certain embodiments, the cluster comprises two or more genes (e.g., 2, 3, 4, 5, 6, 7, 8 or more). In certain embodiments, the cluster is in a transcriptionally active region of the genome. In certain embodiments, the cluster is part of a DNase I hypersensitive site (DHS) and/or unmethylated region of the genome. Methods of assessing DHS of the genome are known in the art, for example, as described in Wenfei Jin et al., Nature, volume 528, pages 142-146 (2015), which is incorporated by reference herein.

Certain conventional GSH may be preferably located at a region (e.g., intergenic region) that does not disrupt a transcriptional unit of the original genomic sequence. However, the synthetic GSH described herein could disrupt a transcriptional unit of the original genomic sequence due to insertion, nonetheless the fitness cost is reduced or eliminated by the inserted synthetic GSH.

Certain conventional GSH may be preferably located at a distance of greater than 50 kb from a transcriptional start site. However, the synthetic GSH described herein is inserted at the locus of an endogenous target gene (e.g., within a transcriptionally active region of genes). In certain embodiments, the synthetic GSH described herein is located within a distance of 50 kb from one or more transcriptional start sites. Namely, if the edited genome sequence having the synthetic GSH is aligned or superimposed with the unedited original genomic sequence, the synthetic GSH described herein can be located within a distance of 50 kb from one or more transcriptional start sites of the original genomic sequence. In certain embodiments, the 5′ end of the synthetic GSH is located within a distance of 50 kb from one or more transcriptional start sites of the original genomic sequence. In certain embodiments, the 3′ end of the synthetic GSH is located within a distance of 50 kb from one or more transcriptional start sites of the original genomic sequence. In certain embodiments, the entire length of the synthetic GSH is located within a distance of 50 kb from one or more transcriptional start sites of the original genomic sequence.

Likewise, certain conventional GSH may be preferably located at a distance of greater than 300 kb from a miRNA gene or at a distance of greater than 100 kb from a lncRNA gene. However, the synthetic GSH described herein could be located close to miRNA or lncRNA gene(s). For example, in certain embodiments, the synthetic GSH described herein is located within a distance of 300 kb, 100 kb, or 50 kb from miRNA or lncRNA gene(s). Namely, if the edited genome sequence having the synthetic GSH is aligned or superimposed with the original genomic sequence, the synthetic GSH described herein can be located within a distance of 300 kb, 100 kb, or 50 kb from miRNA or lncRNA gene(s) of the original genomic sequence. In certain embodiments, the 5′ end of the synthetic GSH is located within a distance of 300 kb, 100 kb, or 50 kb from miRNA or lncRNA gene(s) of the original genomic sequence. In certain embodiments, the 3′ end of the synthetic GSH is located within a distance of 300 kb, 100 kb, or 50 kb from miRNA or lncRNA gene(s) of the original genomic sequence. In certain embodiments, the entire length of the synthetic GSH is located within a distance of 300 kb, 100 kb, or 50 kb from miRNA or lncRNA gene(s) of the original genomic sequence.

As used herein, the term “transgene” refers to a gene that is not natively present at the locus of the endogenous target gene. In certain embodiments, the transgene is an exogenous gene (i.e., a non-native gene that is not present in the genome of the cell) In certain embodiments, the transgene encodes an exogenous protein. In certain embodiments, the transgene is an endogenous gene that is separate and distinct from the target gene (i.e., not an allele of the target gene), thus, the transgene could be ectopically installed at the locus of the target gene as part of the cargo-loaded synthetic GSH, or in the landing pad site (landing sequence) of the receiving synthetic GSH In certain embodiments, the transgene encodes an endogenous protein (e.g., an endogenous wildtype protein). For example, if a host cell has a deficient or mutant gene X on chromosome 1, and the locus of the chosen target gene Y for synthetic GSH insertion is located at chromosome 2 of the host cell, then the synthetic GSH may comprise rescue gene Y and wildtype gene X (WT gene X is the “gene of interest”/transgene to confer benefits to the host cell).

After insertion of synthetic GSH at the locus of the endogenous target gene on the genome, the synthetic GSH could be surrounded by residual vestige sequences of the endogenous target gene that are now separated by the inserted synthetic GSH. In certain embodiments, the synthetic GSH is inserted at an exon sequence of the endogenous target gene of the original genomic sequence. In certain embodiments, the synthetic GSH is inserted at an intron sequence of the endogenous target gene of the original genomic sequence. In certain embodiments, the synthetic GSH is inserted at an exon-intron junction of the endogenous target gene of the original genomic sequence. In certain embodiments, the synthetic GSH is inserted at a regulatory sequence (e.g., promoter or 5′ untranslated region (5′UTR)) of the endogenous target gene of the original genomic sequence. In certain embodiments, the synthetic GSH is inserted at a junction between a regulatory sequence (e.g., promoter or 5′ untranslated region (5′UTR)) and the encoding sequence (e.g., exon 1 and/or intron 1) of the endogenous target gene of the original genomic sequence. In certain embodiments, the synthetic GSH exogenous fusion sequence may be inserted immediately downstream of target gene's promoter and/or 5′-UTR sequence of the endogenous target gene of the original genomic sequence.

In certain embodiments, the exogenous fusion sequence comprises a first flanking sequence that is homologous to an exon. In certain embodiments, the exogenous fusion sequence comprises a second flanking sequence that is homologous to an exon.

In certain embodiments, the exogenous fusion sequence comprises a first flanking sequence that is homologous to an intron. In certain embodiments, the exogenous fusion sequence comprises a second flanking sequence that is homologous to an intron.

In certain embodiments, the exogenous fusion sequence comprises a first flanking sequence that is homologous to an exon. In certain embodiments, the exogenous fusion sequence comprises a second flanking sequence that is homologous to an intron.

In certain embodiments, the exogenous fusion sequence comprises a first flanking sequence that is homologous to a regulatory sequence (e.g., promoter sequence, and/or 5′-UTR sequence). In certain embodiments, the exogenous fusion sequence comprises a second flanking sequence that is homologous to a regulatory sequence (e.g., promoter sequence, and/or 5′-UTR sequence).

In certain embodiments, the exogenous fusion sequence comprises a first flanking sequence that is homologous to a regulatory sequence (e.g., promoter sequence, and/or 5′-UTR sequence). In certain embodiments, the exogenous fusion sequence comprises a second flanking sequence that is homologous to an exon and/or intron sequence.

In certain embodiments, the synthetic GSH exogenous fusion sequence has a length of about 1000, 2000, 3000, 4000, 5000, 6000, 7000, or 8000 nt. In certain embodiments, the synthetic GSH exogenous fusion sequence has a length of about 1000-8000 nt, 2000-8000 nt, 3000-8000 nt, 4000-8000 nt, 5000-8000 nt, 6000-8000 nt, or 7000-8000 nt. In certain embodiments, the synthetic GSH exogenous fusion sequence has a length of about 2000-7000 nt, 3000-7000 nt, 4000-7000 nt, 5000-7000 nt, or 6000-7000 nt. In certain embodiments, the synthetic GSH exogenous fusion sequence has a length of about 2000-6000 nt, 3000-6000 nt, 4000-6000 nt, or 5000-6000 nt. In certain embodiments, the synthetic GSH exogenous fusion sequence has a length of about 1000-5000 nt, 2000-5000 nt, 3000-5000 nt, or 4000-5000 nt.

The size of the minimal, receiving synthetic GSH comprising landing site is dependent on the size of the cDNA (variable with the chosen target gene) and the size of landing sequence having one or more unique sgRNA site. In certain embodiments, two homology arms are included on both ends of the exogenous fusion sequence. For example, in certain embodiments, 5′ homology arm is a sequence having about 1 kb of promoter sequence and 3′ homology arm is a sequence having about 1 kb of an exon, intron, or exon/intron boundary.

A cargo-loaded synthetic GSH can also be assembled without the landing site so that the cargo-loaded GSH comprising rescue gene and transgene(s) can be inserted directly in the genome at the same time.

In certain embodiments, the synthetic GSH is inserted via HDR. In certain embodiments, the synthetic GSH is inserted via nonhomologous end joining (NHEJ).

In certain embodiments, the genome is an insect genome. In certain embodiments, the genome is a bacterial genome. In certain embodiments, the genome is a fungal or oomycete genome. In certain embodiments, the genome is a plant genome. In certain embodiments, the genome is a mammalian genome.

In certain embodiments, the genome is a chromosomal genome. In certain embodiments, the genome is a plasmid genome.

In certain embodiments, the synthetic GSH is inserted into a genome of a cell. In certain embodiments, the synthetic GSH is inserted into a genome of an insect cell. In certain embodiments, the synthetic GSH is inserted into a genome of a mammalian cell. In certain embodiments, the synthetic GSH is inserted into a genome of a bacterial cell. In certain embodiments, the synthetic GSH is inserted into a genome of a fungal or oomycete cell. In certain embodiments, the synthetic GSH is inserted into a genome of a plant cell.

### Methods

Certain embodiments of the invention provide a method of delivering a gene of interest to a cell, or a method of genome editing in a cell, or a method of introducing a synthetic GSH to a cell, the method comprising contacting the cell with a polynucleotide as described herein (e g., an exogenous fusion sequence as described herein).

Certain embodiments of the invention provide a method of making a synthetic GSH (e.g., a minimal receiving sGSH having landing sequence, or a cargo-loaded sGSH having transgene sequence) in a genome of a cell, contacting the cell with a polynucleotide as described herein.

It is possible to make a receiving sGSH first and convert it to a cargo-loaded sGSH by inserting transgene sequence into the landing sequence of receiving sGSH; alternatively, a cargo-loaded sGSH having transgene sequence can be directly made in the genome without making a receiving sGSH first.

Certain embodiments of the invention provide a method of making a synthetic GSH in a genome of a cell, the method comprising:
inserting an exogenous fusion sequence at the locus of an endogenous target gene, wherein the insertion of the fusion sequence inactivates the endogenous target gene, wherein the fusion sequence comprises.
(a) a landing pad comprising gRNA related sequence and PAM site unique to the genome that allows insertion of a transgene sequence encoding a transgene product, and
(b) a complementation sequence comprising a sequence (i.e., a rescue gene sequence) that encodes the target gene product.
In certain embodiments, the method further comprises inserting the transgene sequence encoding the transgene product into the landing pad.

Certain embodiments of the invention provide a method of making a synthetic GSH in a genome of a cell, the method comprising:
inserting an exogenous fusion sequence at the locus of an endogenous target gene, wherein the insertion of the fusion sequence inactivates the endogenous target gene, wherein the fusion sequence comprises:
(a) a transgene sequence encoding the transgene product, and
(b) a complementation sequence comprising a sequence (i.e., a rescue gene sequence) that encodes the target gene product.

Certain embodiments of the invention provide a method of delivering a gene of interest (transgene) to a cell comprising a sGSH described herein, comprising inserting a sequence comprising a transgene sequence encoding the transgene product into a landing sequence of the sGSH, wherein the sGSH comprises an exogenous fusion sequence comprising: (a) a landing sequence described herein, and (b) a complementation sequence comprising a rescue gene sequence that encodes the target gene product as described herein.

Certain embodiments of the invention provide a method of making a synthetic GSH (e.g., a minimal, receiving sGSH) in a genome of a cell, the method comprising:
inserting an exogenous fusion sequence (a first exogenous fusion sequence) at the locus of an endogenous target gene, wherein the insertion of the fusion sequence inactivates the endogenous target gene, wherein the fusion sequence comprises:
(a) a landing sequence described herein, and
(b) a complementation sequence comprising a sequence (i.e., a rescue gene sequence) that encodes the target gene product.

In certain embodiments, a method described herein comprises converting the minimal, receiving sGSH into a cargo-loaded sGSH. For example, the method comprises inserting a second exogenous fusion sequence at the landing sequence, wherein the second fusion sequence comprises a transgene sequence encoding the transgene product.

In certain embodiments, the second fusion sequence further comprises regulatory sequences (e.g., promoter, 5′-UTR, and/or 3′-UTR) as described herein. In certain embodiments, the second fusion sequence comprises a promoter sequence for the transgene as described herein. In certain embodiments, the second fusion sequence comprises 5′-UTR, and/or 3′-UTR sequence(s).

In certain embodiments, the second fusion sequence further comprises two flanking sequences (homology arms upstream and downstream of the transgene sequence).

In certain embodiments, the second fusion sequence comprises a 5′-flanking sequence that is homologous to sequence at the minimal, receiving sGSH. In certain embodiments, the 5′-flanking sequence is homologous to the landing sequence (landing sequence segment upstream of the cutting sequence). In certain embodiments, the 5′-flanking sequence is homologous to a complementation sequence described herein. In certain embodiments, the 5′-flanking sequence is homologous to rescue gene sequence (e.g., last exon). In certain embodiments, the 5′-flanking sequence is homologous to a regulatory sequence, such as a 3′-UTR sequence or a promoter sequence in the minimal, receiving sGSH (e.g., the receiving sGSH may comprise a promoter sequence upstream of the landing sequence and downstream of the complementation sequence).

In certain embodiments, the second fusion sequence comprises a 3′-flanking sequence that is homologous to sequence at the minimal, receiving sGSH. In certain embodiments, the 3′-flanking sequence is homologous to the landing sequence (landing sequence segment downstream of the cutting sequence). In certain embodiments, the 3′-flanking sequence is homologous to endogenous target gene sequence (e.g., downstream segment of the endogenous target gene sequence such as last exon). In certain embodiments, the 3′-flanking sequence is homologous to a regulatory sequence, such as a 3′-UTR sequence of the endogenous target gene sequence.

In certain embodiments, the second fusion sequence comprises two or more transgene sequences encoding two or more transgene products.

As used herein, the term “inactivation of endogenous target gene” refers to the disruption of the transcriptional unit of the endogenous target gene and no intact/functional target gene product could be expressed from the original genomic sequence that encodes the target gene.

In certain embodiments, the complementation sequence is a complementation sequence as described herein.

In certain embodiments, the complementation sequence further comprises a promoter sequence for the rescue gene sequence.

In certain embodiments, the complementation sequence is capable of rescuing the inactivated endogenous target gene. In certain embodiments, the inactivated target gene is rescued by the rescue gene sequence (e.g., comprising full-length cDNA) that encodes the entire target gene product.

In certain embodiments, the method comprises delivering site-specific genome editing enzyme(s) (also referred to as targeted nuclease) to the cell (e.g., delivering CRISPR-Cas enzyme and/or guide RNA to the cell).

Targeted nucleases, and methods of delivery, are known in the art and described herein. In certain embodiment the targeted nuclease is a CRISPR-Cas nuclease (also referred to as a Cas nuclease). In certain embodiments, the Cas nuclease is a CRISPR-Cas9 nuclease or a CRISPR-Cas12a nuclease. In certain embodiments, the Cas9 nuclease is derived from S. pneumoniae, S. pyogenes, S. thermophiles, F. novicida, S. aureus, N. meningitidis, or C. jejuni Cas9, and may include mutations as a Cas9 variant (e.g., Cas9 D10A nickase). In some embodiments, the Cas9 nuclease is SpCas9, SaCas9, StCas9, NmeCas9, or CjCas9. In some embodiments, the Cas12a nuclease is derived from L. bacterium or Acidaminococcus sp. and may include mutations as a Cas12a variant. In some embodiments, the Cas12a nuclease is LpCpf1 or AsCpf1. In certain embodiments, the Cas nuclease is derived from Streptococcus pyogenes Cas9 (e.g., see NCBI Accession NO: WP_010922251).

A guide RNA (e.g., a single guide RNA (sgRNA)) confers target sequence specificity/selectivity for Cas nuclease. Specifically, the guide RNA (gRNA), designed to guide Cas nuclease to cut specific sequence at the locus of the endogenous target gene, complexes with the Cas nuclease and directs cutting at the desired site. gRNA design techniques are described herein and known in the art (see, e.g., U.S. Pat. Nos. 9,790,490; 9,840,702; 9,981,020; 10,106,820 and 10,240,145, which are incorporated by reference herein).

In certain embodiments, the targeted nuclease cuts the original genomic sequence or the landing sequence with a double-stranded DNA break (including blunt end or sticky end).

In certain embodiments, the targeted nuclease cuts the original genomic sequence or the landing sequence with a single-stranded DNA break (e.g., using a nickase).

In certain embodiments, the targeted nuclease cuts the original genomic sequence within an exon sequence of the endogenous target gene. In certain embodiments, the targeted nuclease cuts the original genomic sequence within an intron sequence of the endogenous target gene. In certain embodiments, the targeted nuclease cuts the original genomic sequence at an exon-intron junction of the endogenous target gene. In certain embodiments, the targeted nuclease cuts the original genomic sequence at a regulatory sequence (e.g., promoter or 5′ untranslated region (5′UTR)) of the endogenous target gene. In certain embodiments, the targeted nuclease cuts the original genomic sequence at a junction between a regulatory sequence (e.g., promoter or 5′ untranslated region (5′UTR)) and encoding sequence of the target gene.

In certain embodiments, the method comprises delivering an exogenous fusion sequence described herein to a cell (e.g., a cell having unedited original genome, or a cell having a minimal, receiving sGSH).

In certain embodiments, the method comprises delivering a first exogenous fusion sequence described herein to a cell (e.g., a cell having unedited, original genome). In certain embodiments, the method comprises delivering an exogenous fusion sequence (e.g., a second exogenous fusion sequence) described herein to a cell (e.g., a cell having the receiving sGSH in the genome). In certain embodiments, an exogenous fusion sequence described herein is delivered as single-stranded DNA (ssDNA). In certain embodiments, an exogenous fusion sequence described herein is delivered as double-stranded DNA dsDNA. In certain embodiments, the method comprises delivering a vector (e.g., a plasmid) comprising an exogenous fusion sequence as described herein to the cell. In certain embodiments, the vector (e.g., a plasmid) comprising one or two gRNA sequence(s) that flank the synthetic GSH exogenous fusion sequence as described herein, so that targeted nuclease could cut the gRNA sequence(s) on the vector to release the synthetic GSH exogenous fusion sequence and/or to linearize the vector. In certain embodiments, the method comprises delivering a first vector described herein to a cell (e.g., a cell having unedited, original genome). In certain embodiments, the method comprises delivering a vector described herein (e.g., a second vector) to a cell (e.g., a cell having the receiving sGSH in the genome). In certain embodiments, the method comprises delivering a linearized vector described herein.

In certain embodiments, the method comprises delivering a first targeted nuclease (e.g., a first Cas nuclease/gRNA) described herein to a cell (e.g., a cell having unedited, original genome). In certain embodiments, the method comprises delivering a targeted nuclease (e.g., a second Cas nuclease/gRNA) described herein to a cell (e.g., a cell having the receiving sGSH in the genome).

In certain embodiments, the chosen endogenous target gene has a gRNA sequence that is absent on the synthetic GSH exogenous fusion sequence. For example, the chosen endogenous target gene may have a gRNA sequence at an intron, and the complementation sequence comprises a cDNA sequence for the target gene and therefore does not comprise the intronic sequence targeted by the gRNA/Cas nuclease.

Additionally, the chosen endogenous target gene may have a gRNA sequence at an exon, and the complementation sequence comprises a cDNA sequence comprising alternate codons for the target gene and does not comprise the original exon sequence targeted by the gRNA/Cas nuclease, as long as the complementation sequence comprise a sequence capable of encoding the same target gene product. For example, in certain embodiments, the complementation sequence comprises a rescue gene encoding sequence (e.g., exon(s) and intron(s)) having at least about 85%, 86%, 87%, 88%, 89%, 90%, 91%, 92%, 93%, 94%, 95%, 96%, 97%, 98%, 99% or 100% sequence identity to the native encoding sequence for the endogenous target gene. In certain embodiments, the complementation sequence comprises a cDNA sequence having at least about 85%, 86%, 87%, 88%, 89%, 90%, 91%, 92%, 93%, 94%, 95%, 96%, 97%, 98%, 99%, or 100% sequence identity to the native encoding sequence (such as exon sequence(s), or in mRNA) for the endogenous target gene.

Similarly, the chosen endogenous target gene may have a gRNA sequence at the regulatory sequence (e.g., promoter and/or 5′ UTR), and the complementation sequence may comprise a modified regulatory sequence (e.g., promoter and/or 5′ UTR) that lacks the gRNA sequence targeted by gRNA/Cas nuclease. For example, in certain embodiments, the complementation sequence comprises a promoter sequence (for the rescue gene) having at least about 85%, 86%, 87%, 88%, 89%, 90%, 91%, 92%, 93%, 94%, 95%, 96%, 97%, 98%, 99%, or 100% sequence identity to the native promoter sequence for the endogenous target gene. In certain embodiments, the complementation sequence comprises a 5′ UTR sequence having at least about 85%, 86%, 87%, 88%, 89%, 90%, 91%, 92%, 93%, 94%, 95%, 96%, 97%, 98%, 99% or 100% sequence identity to the native 5′ UTR sequence for the endogenous target gene.

Delivering targeted nuclease and delivering exogenous fusion sequence can be concurrent or sequential. In certain embodiments, delivering targeted nuclease is followed by delivering exogenous fusion sequence. In certain embodiments, delivering exogenous fusion sequence is followed by delivering targeted nuclease.

Deliveries of protein, nucleic acids, complex thereof, and/or vectors into cells are known in the art and are described herein. Targeted nucleases, gRNA, and/or exogenous fusion sequence can be introduced into a cell via lipid-mediated transfection (e.g., cationic lipid), polymer-mediated transfection (e.g., PEG), liposome, nanoparticle, electroporation, microinjection or any suitable methods such as deterministic mechanoporation (DMP) (Nano Lett. 2020 Feb 12; 20(2):860-867). Targeted nucleases can be delivered via intracellular delivery/expression of a vector comprising a nucleic acid encoding the targeted nuclease and/or gRNA. Alternatively, targeted nucleases can be delivered as a protein via intracellular or intranuclear delivery. In certain embodiments, targeted nucleases can be delivered as pre-assembled ribonucleoprotein particles (RNPs) into a cell. For example, Cas nuclease can be mixed with gRNA to form pre-assembled RNPs prior to delivery into a cell.

In certain embodiments, the synthetic GSH is inserted into the genome via homology directed repair (HDR). In certain embodiments, an unedited, original genome is edited into the genome comprising a sGSH by one HDR event with a delivered exogenous fusion sequence or vector described herein. In certain embodiments, a genome having a minimal, receiving sGSH is converted into a genome comprising a cargo-loaded sGSH by one HDR event with a delivered exogenous fusion sequence or vector described herein. In certain embodiments, the synthetic GSH is inserted into the genome via non-homologous end joining (NHEJ).

In certain embodiments, the first exogenous fusion sequence further comprises one or two flanking sequence(s) that are homologous to sequence(s) at the locus of the endogenous target gene. In certain embodiments, the first exogenous fusion sequence does not comprise flanking sequence that is homologous to sequences at the locus of the endogenous target gene.

In certain embodiments, the present invention provides a cell having a genome comprising the synthetic GSH as described herein (e.g., a receiving sGSH having landing sequence, or a cargo-loaded sGSH having transgene). In certain embodiments, the cell is a prokaryotic cell (e.g., a bacterial cell). In certain embodiments, the cell is a fungal or oomycete cell. In certain embodiments, the cell is a eukaryotic cell. In certain embodiments, the cell is a plant cell. In certain embodiments, the cell is an insect cell. In certain embodiments, the cell is a non-mammalian animal cell (e.g., a fish cell). In certain embodiments, the cell is a mammalian cell (e.g., a mouse, rat, dog, cat, monkey, rabbit, hamster, horse, cow, sheep, pig, goat, or camelids cell). In certain embodiments, the cell is a human cell.

In certain embodiments, the present invention provides a non-human organism having a genome comprising the synthetic GSH as described herein (e.g., a receiving sGSH having landing sequence, or a cargo-loaded sGSH having transgene).

In certain embodiments, the organism is a prokaryotic organism (e.g., a bacterium). In certain embodiments, the organism is a fungal or oomycete organism. In certain embodiments, the organism is a eukaryotic organism. In certain embodiments, the organism is a plant. In certain embodiments, the organism is an insect. In certain embodiments, the insect organism is Bemisia tabaci and Homalodisca vitripennis, but the technology can be applied to any insect species amenable to gene editing.

In certain embodiments, the insect organism is from the order Diptera, Lepidoptera, Coleoptera, Hemiptera, or Orthoptera. In certain embodiments, the insect organism is from the Aleyrodidae family. In certain embodiments, the insect organism is a psyllid, sharpshooter, leafhopper, planthopper, aphid, Bagruda bug, Lygus bug, box elder bug, chili thrip, crape myrtle bark scale, four-lined plant bug, pink hibiscus mealybug, scale insect, cycad aulacaspis scales, or wax scales on holly. In certain embodiments, the insect is not a mosquito.

In certain embodiments, the organism is a non-mammalian organism (e.g., a fish). In certain embodiments, the organism is a mammalian organism (e.g., a mouse, rat, dog, cat, monkey, rabbit, hamster, horse, cow, sheep, pig, goat, or camelids cell). In certain embodiments, the organism is a non-human organism.

### Certain Definitions

The term “nucleic acid” and “polynucleotide” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

A “variant” of a molecule is a sequence that is substantially similar to the sequence of the native molecule.

“Recombinant nucleic acid molecule” is a combination of nucleic acid sequences that are joined together using recombinant nucleic acid technology and procedures used to join together nucleic acid sequences as described, for example, in Sambrook and Russell (2001), Gibson et al. Nature Methods. 6 (5): 343-345. (2009). As used herein, the term “recombinant nucleic acid,” e.g., “recombinant DNA sequence or segment” refers to a nucleic acid, e.g., to DNA, that has been derived or isolated from any appropriate cellular source, that may be subsequently chemically altered in vitro, so that its sequence is not naturally occurring, or corresponds to naturally occurring sequences that are not positioned as they would be positioned in a genome that has not been transformed with exogenous DNA. An example of preselected DNA “derived” from a source would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases or the polymerase chain reaction (PCR), so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. Therefore, “recombinant DNA” includes completely synthetic DNA sequences, semi-synthetic DNA sequences, DNA sequences isolated from biological sources, and DNA sequences derived from RNA, as well as mixtures thereof.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least about one exon and (optionally) an intron sequence.

A “vector” is defined to include, inter alia, any plasmid, cosmid, phage, or binary vector in double- or single-stranded linear or circular form which may or may not be self-transmissible or mobilizable, and which can transform a host cell either by integration into the cellular genome or exist extrachromosomally (e.g., autonomous replicating plasmid with an origin of replication).

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least about one of its components is heterologous with respect to at least about one of its other components. The expression cassette may also be one that is naturally occurring but has been obtained in a recombinant form useful for heterologous expression. The expression of the nucleotide sequence in the expression cassette may be under the control of a constitutive promoter, developmentally regulated, tissue or cell specific promoter, or of an inducible promoter that initiates transcription only when the host cell is exposed to some particular external stimulus.

The term “RNA transcript” or “transcript” refers to the product resulting from RNA polymerase catalyzed transcription of a DNA sequence. When the RNA transcript is a perfect complementary copy of the DNA sequence, it is referred to as the primary transcript, or it may be an RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” are nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, development-specific promoters, regulatable promoters, and viral promoters.

“5′-UTR (non-coding sequence)” or “5′-untranslated region” refers to a nucleotide sequence located 5′ (upstream) to the coding sequence. It is present in the fully processed mRNA upstream of the initiation codon and may affect processing of the primary transcript to mRNA, mRNA stability or translation efficiency.

“3′-UTR (non-coding sequence)” or “3′-untranslated region” refers to nucleotide sequences located 3′ (downstream) to a coding sequence and may include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

“Promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

“Expression” refers to the transcription and/or translation of an endogenous gene, heterologous gene or nucleic acid segment, or a transgene in cells. Expression may also refer to the production of protein.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

As used herein, the term “operably linked” refers to a linkage of two elements in a functional relationship. For example, “operably linked” may refer to a linkage of polynucleotide elements or polypeptide elements in a functional relationship. A nucleic acid is “operably linked” when it is placed into a functional relationship with another nucleic acid sequence. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. “Operably-linked” also refers to the association two chemical moieties so that the function of one is affected by the other, e.g., an arrangement of elements wherein the components so described are configured so as to perform their usual function.

The term “amino acid” includes the residues of the natural amino acids (e.g., Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g., dehydroalanine, homoserine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g., acetyl or benzyloxycarbonyl), as well as natural and unnatural amino acids protected at the carboxy terminus (e.g., as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein) The term also comprises natural and unnatural amino acids bearing a cyclopropyl side chain or an ethyl side chain.

The terms “polypeptide” and “protein” are used interchangeably herein. A protein molecule may exist in a purified form or may exist in a non-native environment such as, for example, a transgenic host cell or bacteriophage. Fragments and variants of the disclosed proteins or partial-length proteins encoded thereby are also encompassed by the present invention. By “fragment” or “portion” is meant a full length or less than full length of the amino acid sequence of a protein.

By “portion” or “fragment,” as it relates to a nucleic acid molecule, sequence or segment of the invention, when it is linked to other sequences for expression, is meant a sequence having at least about 80 nucleotides, more preferably at least about 150 nucleotides, and still more preferably at least about 400 nucleotides. If not employed for expressing, a “portion” or “fragment” means at least about 9, preferably 12, more preferably 15, even more preferably at least about 20, consecutive nucleotides, e.g., probes and primers (oligonucleotides), corresponding to the nucleotide sequence of the nucleic acid molecules of the invention.

The invention encompasses isolated or substantially purified protein compositions. In the context of the present invention, an “isolated” or “purified” polypeptide is a polypeptide that exists apart from its native environment and is therefore not a product of nature. A polypeptide may exist in a purified form or may exist in a non-native environment such as, for example, a transgenic host cell. For example, an “isolated” or “purified” protein, or biologically active portion thereof, is substantially free of other cellular material, or culture medium when produced by recombinant techniques, or substantially free of chemical precursors or other chemicals when chemically synthesized. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of- interest chemicals. Fragments and variants of the disclosed proteins or partial-length proteins encoded thereby are also encompassed by the present invention. By “fragment” or “portion” is meant a full length or less than full length of the amino acid sequence of, a polypeptide or protein.

The terms “introduce to a cell” and “delivery to a cell” refers to contacting a cell with a composition described herein for intracellular delivery or administration of the composition. The delivered components can be provided as isolated or purified protein, nucleic acids (such as DNA or RNA), a vector, or any combination thereof. Thus, the methods of introduction or delivery can be a combination of delivery methods. For example, a polypeptide or an RNA can be introduced via intracellular delivery/expression of a vector comprising a nucleic acid encoding the recombinant polypeptide or the RNA. Non-limiting examples of vector delivery methods include transformation (e.g., transduction), viral and non-viral based delivery, nanoparticle delivery, liposomal delivery, etc. Alternatively, polypeptide(s) and nucleic acids can be introduced through the use of non-limiting examples of nanoparticles, liposomes, electroporation, microinjection, and gene gun, etc.

The term “transformation” refers to the transfer of a nucleic acid fragment into the genome of a host cell, resulting in genetically stable inheritance. A “host cell” is a cell that has been transformed, or is capable of transformation, by an exogenous nucleic acid molecule. Host cells containing the transformed nucleic acid fragments are referred to as “transgenic” cells.

“Transformed,” “transduced,” “transgenic” and “recombinant” refer to a host cell into which a heterologous nucleic acid molecule has been introduced. The term “transformation” is used herein to refer to delivery of DNA into prokaryotic (e.g., E. coli) cells. The term “transduction” is used herein to refer to infecting cells with viral particles. The nucleic acid molecule can be stably integrated into the genome generally known in the art. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “comparison window” makes reference to a contiguous and specified segment of an amino acid or polynucleotide sequence, wherein the sequence in the comparison window may comprise additions or deletions (i.e., gaps) compared to the reference sequence (which does not comprise additions or deletions) for optimal alignment of the two sequences. Generally, the comparison window is at least about 20 contiguous amino acid residues or nucleotides in length, and optionally can be 30, 40, 50, 100, or longer.

As used herein, “percentage of sequence identity” means the value determined by comparing two optimally aligned sequences over a comparison window, wherein the portion of the polypeptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least about 70%, 71%, 72%, 73%, 74%, 75%, 76%, 77%, 78%, or 79%, at least about 80%, 81%, 82%, 83%, 84%, 85%, 86%, 87%, 88%, or 89%, at least about 90%, 91%, 92%, 93%, or 94%, and at least about 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least about 70%, at least about 80%, 90%, or at least about 95%.

The term “substantial identity” in the context of a peptide indicates that a peptide comprises a sequence with at least about 70%, 71%, 72%, 73%, 74%, 75%, 76%, 77%, 78%, or 79%, 80%, 81%, 82%, 83%, 84%, 85%, 86%, 87%, 88%, or 89%, at least about 90%, 91%, 92%, 93%, or 94%, or 95%, 96%, 97%, 98% or 99%, sequence identity to the reference sequence over a specified comparison window. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity or complementarity for the test sequence(s) relative to the reference sequence, based on the designated program parameters.

The invention will now be illustrated by the following non-limiting Examples.

### EXAMPLE 1

### Introduction

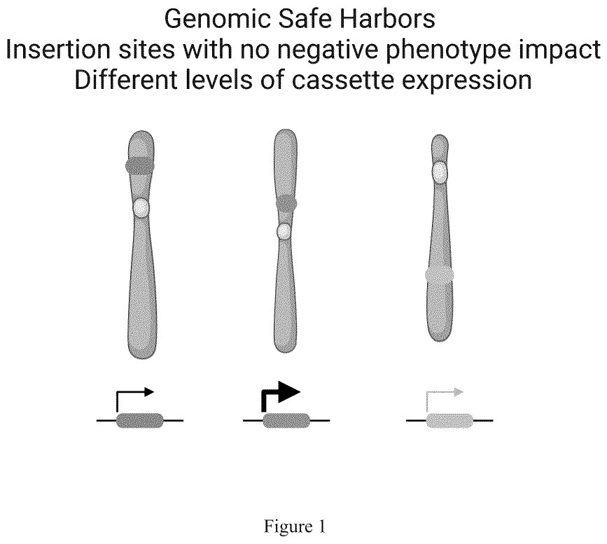

Integration of transgenes into a genome often leads to low levels of gene expression or gene inactivation (FIG. 1). Such events limit genetic strategies. Optimal genome sites for expressing transgenes are important in insect gene-drive control strategies, insect sterile-release control programs, transgenic plants designed to express genes for insect control, human cell and gene therapies, and for expression of proteins for medicine, industry and nutrition. Hence investigators have sought optimal genome sites for transgene insertion.

Genomic safe harbors (GSHs) are sites within an organism's genome, where transgenes can be inserted without negative fitness costs, promoting stable and high-level transgene expression, and having a low probability of silencing. The vast majority of GSH papers focus on the identification and use of GSHs for transgene expression for human cell and gene therapies and production of therapeutic proteins (Papapetrou and Schambach 2016a; Yamamoto and Gerbi 2018). GSHs are most often identified by large-scale screens using insertional mutagenesis of mammalian cells in culture or by T-DNA or transposon mutant collections of plants (Papapetrou and Schambach 2016a; Rozov et al. 2022; Dong et al. 2020). For agribusiness, hundreds of transgenic plants must be screened for the transgene in an insertion site that allows for optimal expression; this must be followed by rigorous biochemical characterization to assure that there are no off-target impacts or fitness costs.

While important for gene-drive strategies for insect control, the importance of the site for gene-drive cassette insertion is only now beginning to be acknowledged. In fact, there is only one report of an insect GSH. Cell cultures of the sleeping chironomid, Polypedilum vanderplanki (Diptera) were screened for high level expression of transgenes. (Miyata et al. 2022a). Four GSHs were identified; three were in intergenic regions and one was within an intron. The larger genomic context (i.e., proximity to transposons, other repetitive elements or chromatin status) was not reported.

Computational approaches have also been used to identify potential GSHs (Autio et al. 2021; Furukawa et al. 2022; Aznauryan et al. 2022). Certain criteria for putative GSHs have been set for human gene therapies and some of these criteria may be useful for the identification of potential GSHs in insects. These putative GSHs should: (1) be >50 kb from a transcriptional start site, (2) not disrupt a transcriptional unit, (3) be >300 kb from miRNAs or >100 kb from IncRNAs, (4) be located outside of DNase I hypersensitivity clusters, which are likely enriched for binding sites for regulatory factors, and (5) be outside of ultra-conserved regions of the genome (Papapetrou and Schambach 2016a). Finally, GSHs should promote stable gene expression of transgenes in all tissue types across multiple generations.

For non-model organisms, the genomics resources (i.e., multiple annotated genomes, a chromosomal level genome assembly, large numbers of transcriptomes from different organs or populations, a knowledge of chromatin accessibility, and location of miRNAs and IncRNAs) needed for computational identification of GSHs are largely lacking. Arras et al., (2015) working with the yeast Cryptococcus neoformans identified two criteria for GSHs: that they be flanked by convergently transcribed genes and that they be in one of the larger intergenic regions. C. neoformans has a very compact genome and so the lengths of intergenic regions very small relative to those of insects. Furthermore, most non-model insects do not have insertional mutant collections or cell culture lines that enable high-throughput screens for GSH identification. At least for this reason, identifying GSHs in non-model insects is challenging, but remains critical for the successful deployment of sustainable gene-drive strategies. For example, at present, there are only two reports of gene drive in an insect species outside the Order Diptera, and both are in the diamondback moth, Plutella xylostella, a major pest of international agriculture (Asad et al. 2022; Xu et al. 2022). Gene drive was weak in the study by Asad et al (2022) and not observed in the study of Xu et al (2022). Xu et al. (2022) suggested that safe harbors for gene-drive cassette insertion should be sought. GSHs are also important for our proposed methods of insect control that rely on transgene expression of double-stranded RNAs or Cas9 and sgRNAs in plants. For this reason, we propose a novel method for making synthetic GSHs, we call Target-on-Demand (ToD). We discuss the ToD concept in the context of insect gene drive. However, it could have wide ranging impact on mammalian, plant and insect biotechnology.

Our approach is based on gene complementation. The ability of a wild-type cDNA to substitute for the mutated gene and, simultaneously, tailor transcription of the transgene to the desired tissues and levels.

### Synthetic Genomic Safe Harbor (GSH) and Target-on-Demand (ToD) Approach

The site of integration of transgene cassettes influences the sustainability and effectiveness of a gene drive and its level of expression. Surprisingly, while acknowledged as an important feature for successful transgenesis and gene drive, GSHs have received relatively little attention in the insect gene-drive literature or in the insect community overall. This is due to the fact that identification of GSHs in non-model organisms has few precedents. Described herein are exemplary methods to create a synthetic GSH that can transform “any” gene into a GSH and so increases dramatically the number of sites that can be identified and tested (a targets on demand, ToD).

To date, genome insertion site targets for gene-drive cassettes have been genes with an easily identified morphological phenotypes, such as eye pigmentation or body color. When used as target genes in mosquitoes, eye-color genes w and cn exhibit different gene drive efficiencies, 59% and 38%, respectively. Moreover, in our hands, w and cn mutations have a mild fitness cost impacting the success of Glassy-winged sharpshooter (GWSS) paired matings (but, luckily, not pool matings). Therefore, these genes are not GSHs in GWSS. In whitefly, w mutations are lethal. It is clear new gene-drive insertion sites are needed.

Therefore, to enable a robust and sustainable GWSS or whitefly gene drive, we need to identify GSH loci as a landing and launching pads for gene drive in these insects. Optimal target sites are also needed for the insertion of genes for sterile insect control programs.

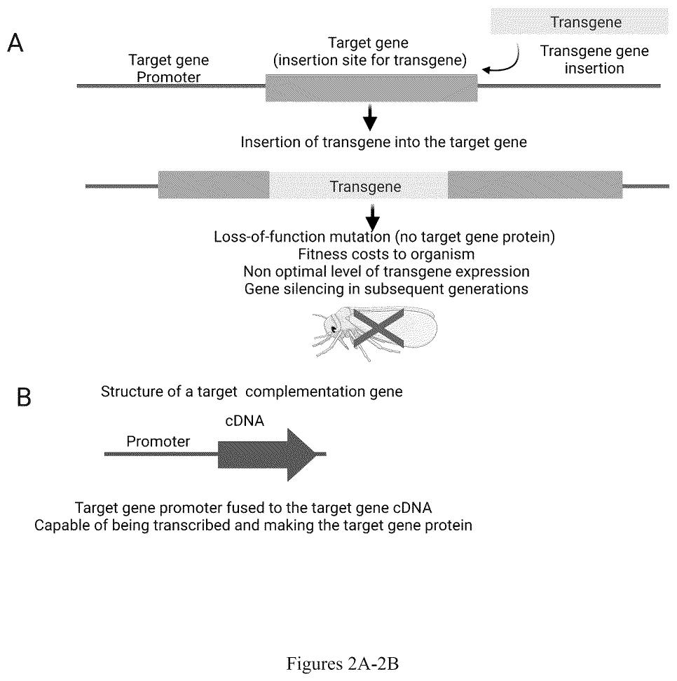

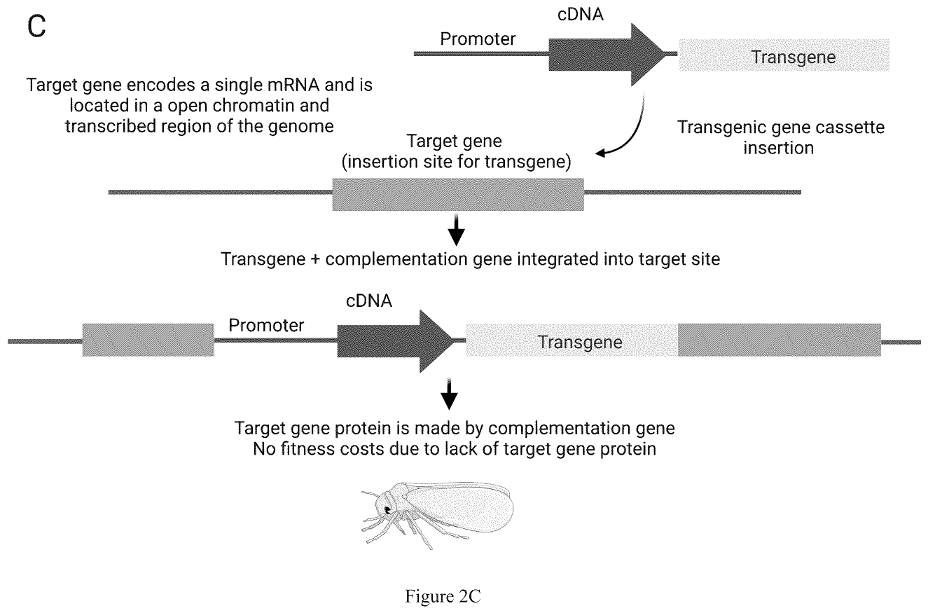

For non-model organisms, a simple and yet widely applicable method for creating a GSH would revolutionize our ability to express gene products and develop durable gene drives. Described herein is an exemplary method to custom design a synthetic GSH—a target on demand (FIG. 2). In this manner, virtually “any” gene can become a GSH. In certain embodiments, such a gene should reside in a transcriptionally active region and not use alternative splicing as a mechanism of gene regulation. This strategy is simple, since the loss-of-function insertion of a cassette into a gene often has a fitness cost (FIG. 2A), methods described herein complement the loss-of-function mutation with a chimeric gene (FIG. 2B-2C).

The ToD scheme is illustrated with an experimental design using the GWSS cn gene (FIG. 3). The GWSS cn is not a GSH, as cn mutants have mild fitness costs that interfere with pair matings. However, we can integrate genes into the GWSS cn with high efficiency using HDR and CRISPaint. The cn deficiency caused by a cassette insertion is complemented by providing a cn complementation gene (FIG. 3B-3C). In this example, the proof-of-concept complementation cassette has a reporter gene (dsRed) that produces a red fluorescent protein that allows us to follow cassette integration into cn by monitoring fluorescence. In this example, the complementation gene is the cn cDNA expressed using its native 3-kb cn promoter (Cn:cn-cDNA). In addition, 1-kb cn homology arms are used for efficient integration of the ToD cassette into the cn gene by HDR. The ToD-cn plasmid, sgRNA-cn and Cas9 are microinjected in GWSS embryos. G0 embryos and nymphs are screened for dsRED fluorescence and eye color (FIG. 3).

Four possible G0 phenotypic classes could be generated: mosaic cn⁻ eyes, mosaic cn⁻ eyes and dsRED fluorescence, wild-type eyes, wild-type eyes and dsRED fluorescence. Insects in each class are pooled and virgin adults from this pool are pair mated. G0 insects that have wild-type eyes (cn⁺) and are dsRed⁺ (phenotype indicative of success) should be the result of complementation. Paired matings of these insects should have rates of egg hatch similar to wild-type insects, further indicating the success of complementation using the ToD strategy. Whereas cn−/dsRed⁺ insects should yield no progeny from pair matings; they would represent a failure of complementation.

### References in Example 1

Arras SDM, Chitty JL, Blake KL, Schulz BL, Fraser JA (2015) A genomic safe haven for mutant complementaton in Crytococcus neoformans. PLOS One 10(4):e0122916.doi:10.1371/journal.pone.0122916.
Asad M, Liu D, Li J, Chen J, Yang G (2022) Development of CRISPR/Cas9-Mediated Gene-Drive Construct Targeting the Phenotypic Gene in Plutella xylostella. Frontiers in Physiology 13. doi:10.3389/fphys.2022.938621
Autio M I, Motakis E, Perrin A, Bin Amin T, Tiang Z, Do D V, Wang J, Tan J, Tan W X, Ding S, Teo A K K, Foo RSY (2021) Computationally defined human genomic safe harbour loci validated in vitro for stable transgene expression. Human Gene Therapy 32 (19-20):A67-A68
Aznauryan E, Yermanos A, Kinzina E, Devaux A, Kapetanovic E, Milanova D, Church GM, Reddy ST (2022) Discovery and validation of human genomic safe harbor sites for gene and cell therapies. Cell Reports Methods 2 (1): 100154. doi:https://doi.org/10.1016/j.crmeth.2021.100154
Dong OXO, Yu S, Jain R, Zhang N, Duong PQ, Butler C, Li Y, Lipzen A, Martin JA, Barry KW, Schmutz J, Tian L, Ronald PC (2020) Marker-free carotenoid-enriched rice generated through targeted gene insertion using CRISPR-Cas9. Nature Communications 11 (1). doi:10.1038/s41467-020-14981-y
Furukawa T, van Rhijn N, Chown H, Rhodes J, Alfuraiji N, Fortune-Grant R, Bignell E, Fisher MC, Bromley M (2022) Exploring a novel genomic safe-haven site in the human pathogenic mould Aspergillus fumigatus. Fungal Genet Biol 161:103702. doi:10.1016/j.fgb.2022.103702
Miyata Y, Tokumoto S, Arai T, Shaikhutdinov N, Deviatiiarov R, Fuse H, Gogoleva N, Garushyants S, Cherkasov A, Ryabova A, Gazizova G, Cornette R, Shagimardanova E, Gusev O, Kikawada T (2022) Identification of Genomic Safe Harbors in the Anhydrobiotic Cell Line, Pv11. Genes 13 (3). doi:10.3390/genes13030406
Papapetrou EP, Schambach A (2016) Gene Insertion Into Genomic Safe Harbors for Human Gene Therapy. Mol Ther 24 (4):678-684. doi:10.1038/mt.2016.38
Rozov SM, Permyakova NV, Sidorchuk YV, Deineko EV (2022) Optimization of Genome Knock-In Method: Search for the Most Efficient Genome Regions for Transgene Expression in Plants. International Journal of Molecular Sciences 23 (8). doi:10.3390/ijms23084416
Xu X, Harvey-Samuel T, Siddiqui HA, De Ang JX, Anderson ME, Reitmayer CM, Lovett E, Leftwich PT, You M, Alphey L (2022) Toward a CRISPR-Cas9-based gene drive in the diamondback moth Plutella xylostella. The CRISPR Journal 5 (2):224-236. doi:10.1089/crispr.2021.0129
Yamamoto Y, Gerbi SA (2018) Making ends meet: targeted integration of DNA fragments by genome editing. Chromosoma 127 (4):405-420. doi:10.1007/s00412-018-0677-6

### EXAMPLE 2

### Introduction

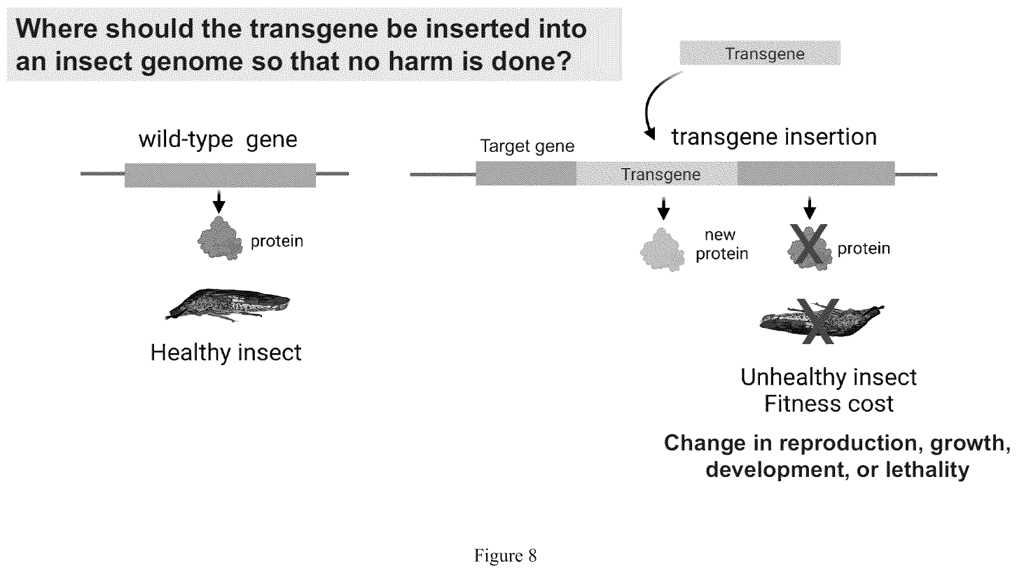

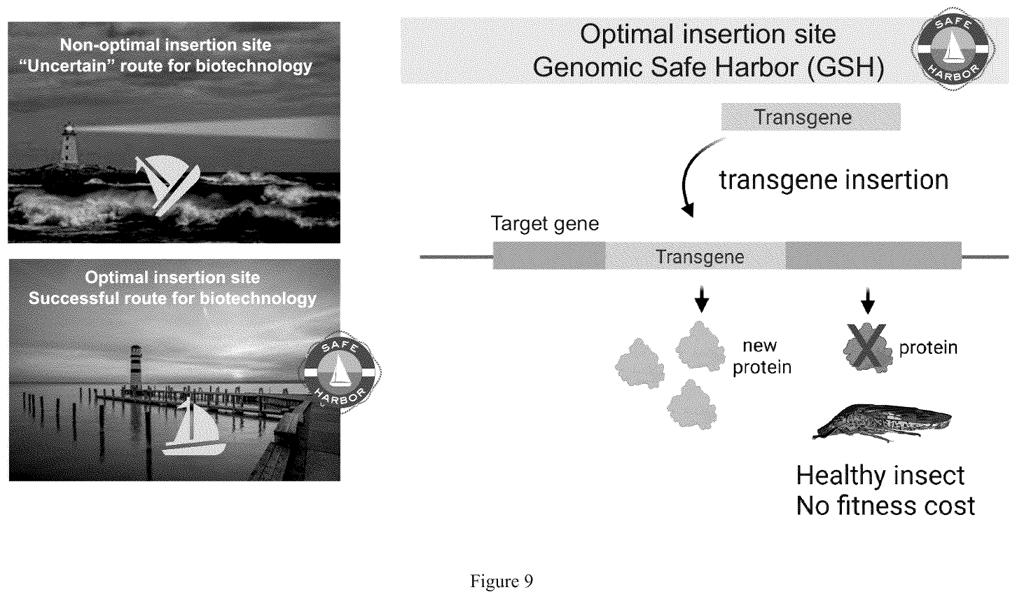

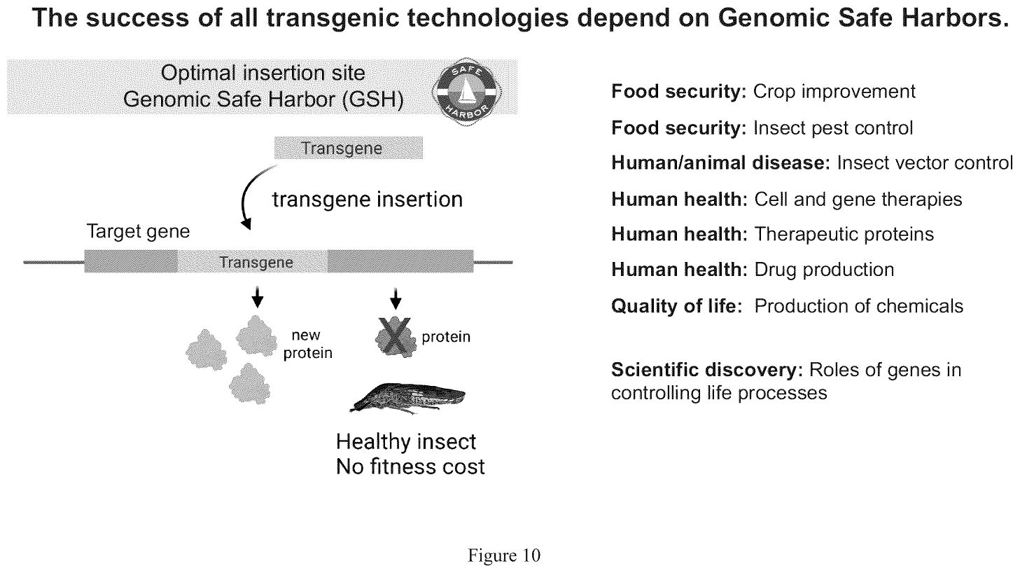

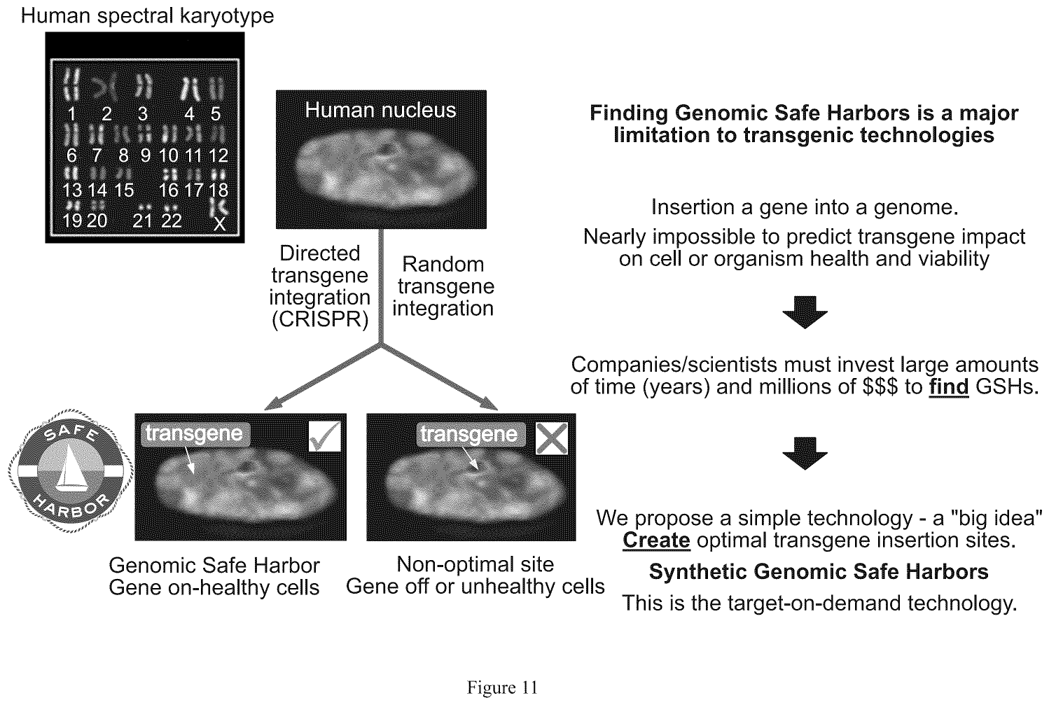

Integration of transgenes into a genome often leads to low levels of gene expression or gene inactivation (FIG. 1, and FIG. 8). Such events limit genetic strategies. Optimal genome sites for expressing transgenes (FIG. 9) are important in insect gene-drive control strategies, insect sterile-release control programs, transgenic plants designed to express genes for insect control, human cell and gene therapies, and for expression of proteins for medicine, industry and nutrition. Hence investigators have sought optimal genome sites for transgene insertion.

Genomic safe harbors (GSHs) are sites within an organism's genome, where transgenes can be inserted without negative fitness costs, promoting stable and high-level transgene expression, and having a low probability of silencing. The vast majority of GSH papers focus on the identification and use of GSHs for transgene expression for human cell and gene therapies and production of therapeutic proteins (Papapetrou and Schambach 2016a; Yamamoto and Gerbi 2018). GSHs are most often identified by large-scale screens using insertional mutagenesis of mammalian cells in culture or by neutron particle, T-DNA or transposon mutant collections of plants (Papapetrou and Schambach 2016a; Rozov et al. 2022; Dong et al. 2020). For agribusiness, hundreds of transgenic plants must be screened for the transgene in an insertion site that allows for optimal expression; this must be followed by rigorous biochemical characterization to assure that there are no off-target impacts or fitness costs.

While important for gene-drive strategies for insect control, the importance of the site for gene-drive cassette insertion is only now beginning to be acknowledged (Xu et al, 2012). In fact, there is only one report of an insect GSH. Cell cultures of the sleeping chironomid, Polypedilum vanderplanki (Diptera) were screened for high level expression of transgenes. (Miyata et al. 2022a). Four GSHs were identified; three were in intergenic regions and one was within an intron. The larger genomic context (i.e., proximity to transposons, other repetitive elements or chromatin status) was not reported.

### Current Methods for GSH Identification

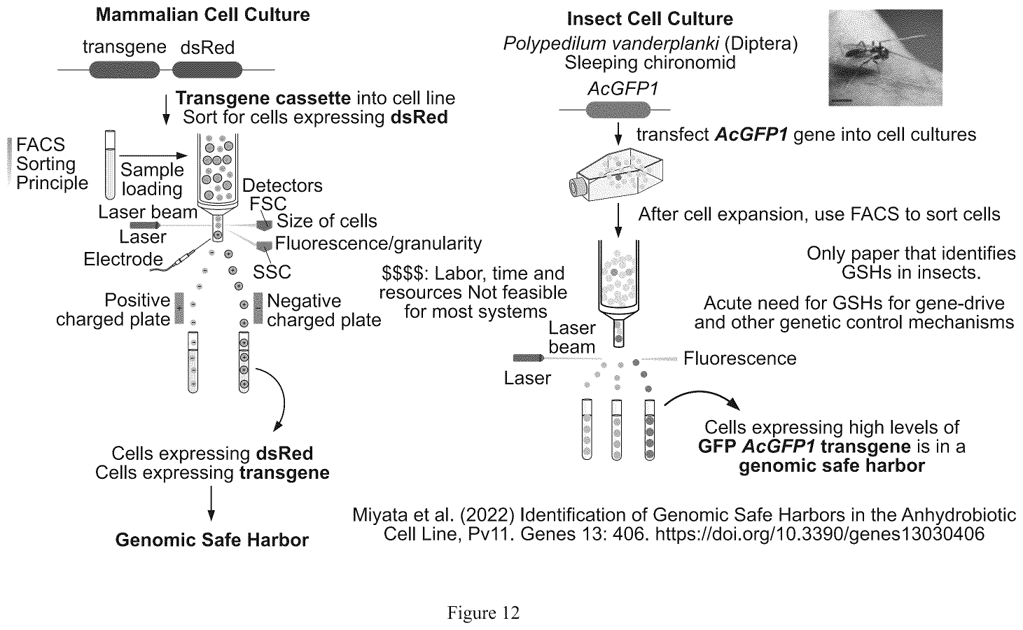

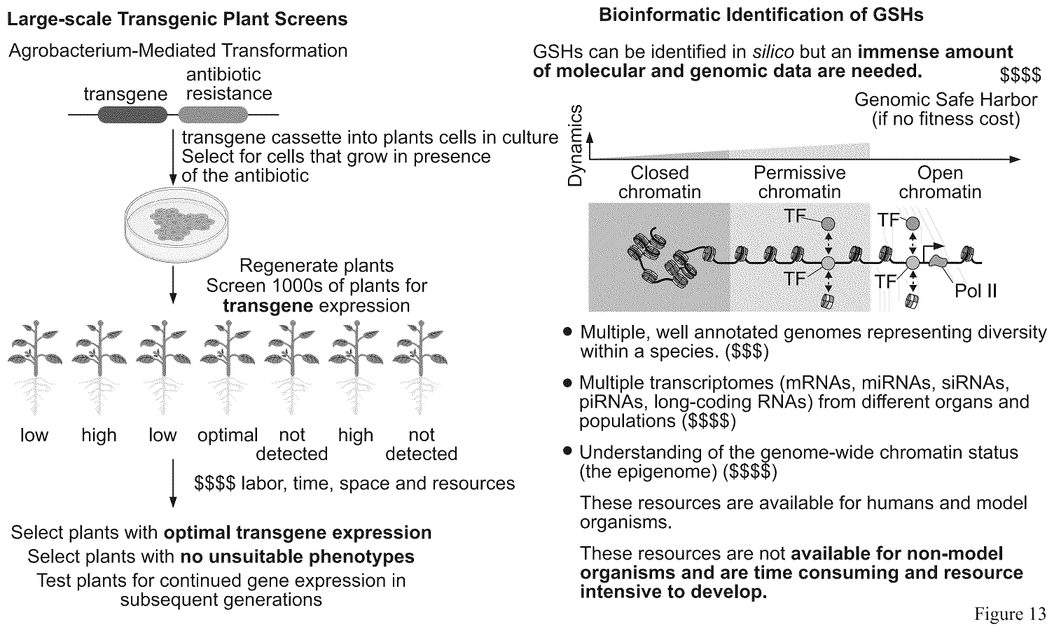

To date, there are relatively few strategies that have been used to identify GSHs and all are labor-intensive. These strategies have relied on: (1) large screens of transgene expression cells in culture (in mammals and insects) (FIG. 12); (2) large screen of transgenic plants for optimal lines; and (3) computational approaches (FIG. 13).

In mammals and insects, cell cultures are used to identify GSHs. Transgenic cells are sorted to identify cells expressing a fluorescent reporter gene at high levels inferring a GSH (FIG. 12) (Miyata et al. 2022b). In insects, there is only one report of a GSH. Cell cultures of the sleeping chironomid, Polypedilum vanderplanki (Diptera) were screened for high-level expression of transgenes (Miyata et al. 2022b). Four GSHs were identified; three were in intergenic regions and one was within an intron. The larger genomic context (i.e., proximity to transposons, other repetitive elements or chromatin status) was not reported. In plants, large numbers of transgenic plants are screened to identify plants with transgene insertions. Depending on the trait and tissue, expression of the transgene may need to be expressed in organs of mature plants (FIG. 13).

An alternative strategy was used to identify GSHs in rice. In this case, morphological records and the whole-genome sequencing data of a fast-neutron rice mutant collection was surveyed and five mutant loci were identified with no apparent fitness costs (Li et al. 2017; Jung et al. 2008). These loci were tested for use as GSHs and one allowed stable expression of a 5.2-kb transgene cassette that promoted carotenoid production (Dong et al. 2020).

Computational approaches have also been used to identify potential GSHs (Autio et al. 2021; Furukawa et al. 2022; Aznauryan et al. 2022; Arras et al. 2015; Balmas et al. 2023; Dabiri et al. 2023; Ittiprasert et al. 2023). In these studies, the foremost concerns are to assure that GSHs will promote stable gene expression of transgenes (e.g., in all tissue types) across multiple generations and transgenes will not directly or indirectly impact potential cancer-inducing genes (Dabiri et al. 2023). About eight criteria have been iterated for bioinformatic identification of putative GSHs (Papapetrou et al. 2011; Chekulaeva and Filipowicz 2009; Van Meter et al. 2020; Dabiri et al. 2023; Papapetrou and Schambach 2016b; Odak et al. 2020). To assure that the transgene does not inactivate a critical gene or regulatory element (e.g., small RNAs) and is not influenced by regional enhancers, silencers or insulators, people have proposed that a GSH should be: >50 kb from a transcriptional start site (1st criterion); not disrupt a transcriptional unit (2nd criterion); be >300 kb from miRNAs (3rd criterion); be >300 kb from known cancer-associated genes (4th criterion); >100 kb from non-coding RNAs (eg., lncRNAs) (5th criterion); and be outside of ultra-conserved regions of the genome (Papapetrou and Schambach 2016a), which may harbor essential genes or structural elements (6th criterion). Additionally, to assure that a transgene is expressed at desired levels and is not silenced in subsequent generations, GSHs should be located in open chromatin domains to allow transgene expression (7th criterion) and easy access of DNA-cutting enzymes critical for gene insertion (8th criterion).

For non-model organisms, the genomics resources (i.e., multiple annotated genomes, a chromosomal level genome assembly, large numbers of transcriptomes from different organs or populations, a knowledge of chromatin accessibility, and location of miRNAs and IncRNAs) needed for computational identification of GSHs are largely lacking. Collecting these deep genomic resources is costly and time consuming and not feasible for many non-model organisms. Most non-model organisms do not have cell cultures that allow for large scale screens to identify GSHs (FIG. 12 and FIG. 13) and some scientists question the value of screens in cell culture vs intact organisms. Furthermore, most non-model insects do not have insertional mutant collections or cell culture lines that enable high-throughput screens for GSH identification (Dong et al. 2020; Jeong et al. 2023; Malaiwong et al. 2023).

Therefore, alternative criteria have been used in some non-model organisms. For example, two criteria for GSHs were used for identifying GSHs in the yeast Cryptococcus neoformans, which has a compact genome and short intergenic regions (Arras et al. 2015) relative to those of insects. GSHs are flanked by convergently transcribed genes (criterion 1) and in a large intergenic region (criterion 2). Other non-model organisms have also stressed the need for GSHs. Approaches have included: (1) testing GSH regions identified in other organisms (i.e, ROSA26, AAVS1, H11 and COLIA1) in chickens (Ma et al. 2022), (2) combining chromatin accessibility (epigenome) and genome resources in blood flukes (Ittiprasert et al. 2023), (3) using epi/genome resources and a large scale screen of Cas9 mutational hotspots in microalgae (Jeong et al. 2023), and (4) leveraging the serendipitous discovery of a TGFβ receptor 2-like gene in Xenopus as a safe harbor (Shibata et al. 2023; Shibata et al. 2022).

Overall, identifying GSHs in model and non-model insects is challenging, but remains important for the successful deployment of sustainable gene-drive strategies. At present, there are only two reports of gene drive in an insect species outside the Order Diptera, and both are in the diamondback moth, Plutella xylostella, a major pest of international agriculture (Asad et al. 2022; Xu et al. 2022). Gene drive was weak in the study by Asad et al (2022) and not observed in the study of Xu et al (2022). Xu et al. (2022) suggested that genomic safe harbors (GSHs) for gene-drive cassette insertion should be sought. GSHs are also important for our proposed methods of insect control that rely on transgene expression of double-stranded RNAs or Cas9 and sgRNAs in plants.

### Synthetic Genomic Safe Harbor (GSH) and Target-on-Demand (ToD)) Approach

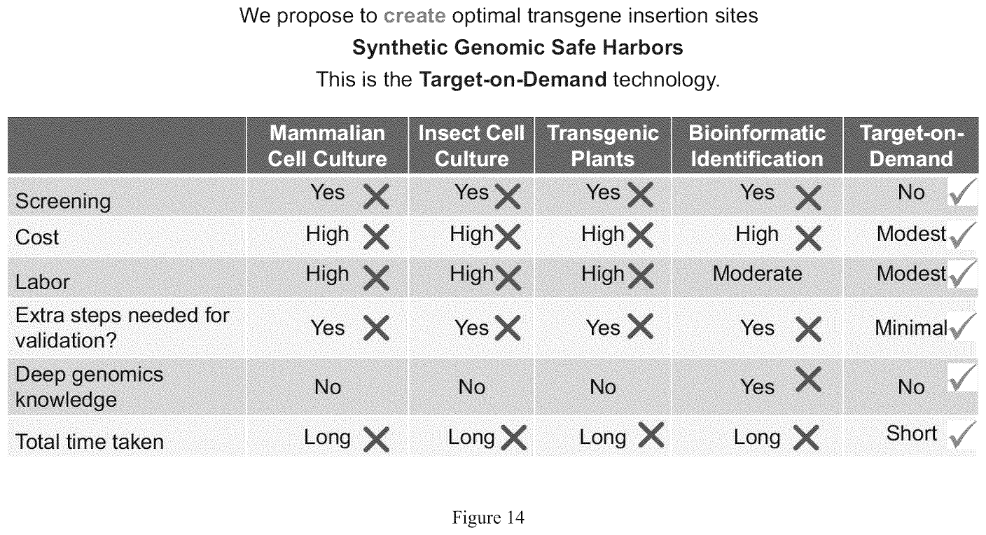

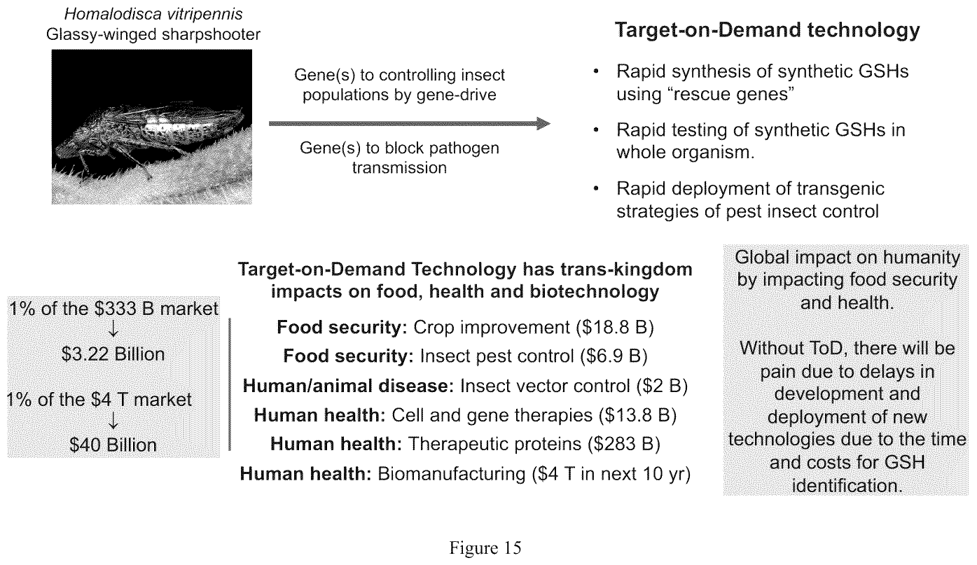

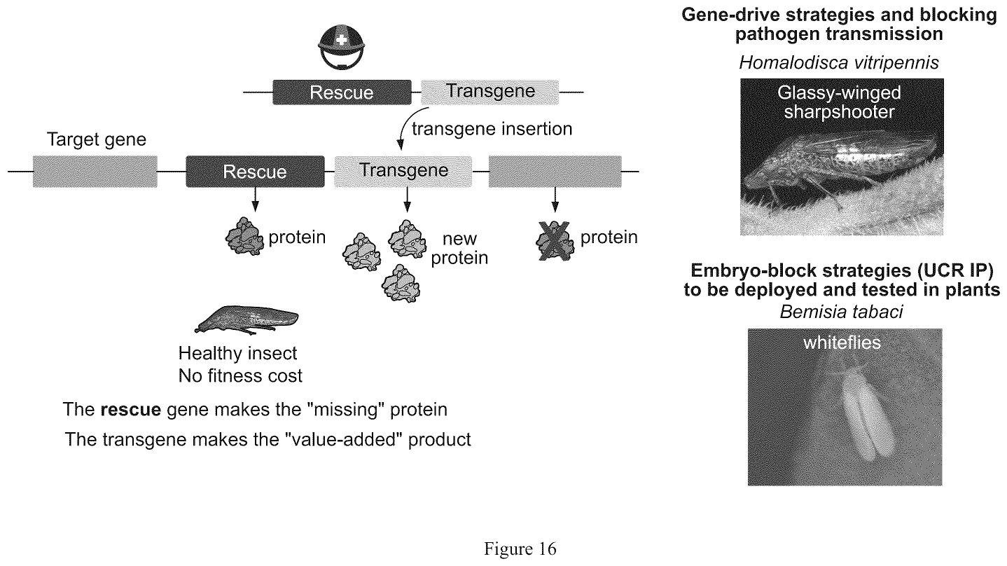

The site of integration of transgene cassettes influences the sustainability and effectiveness of a gene drive and its level of expression. Surprisingly, while acknowledged as an important feature for successful transgenesis and gene drive, GSHs have received relatively little attention in the insect gene-drive literature or in the insect community overall. This is due to the fact that identification of GSHs in non-model organisms has few precedents. For this reason, we propose a novel method for making synthetic GSHs, we call Target-on-Demand (ToD). A competitive matrix of the current and our proposed method for GSH discovery is provided in FIG. 14.

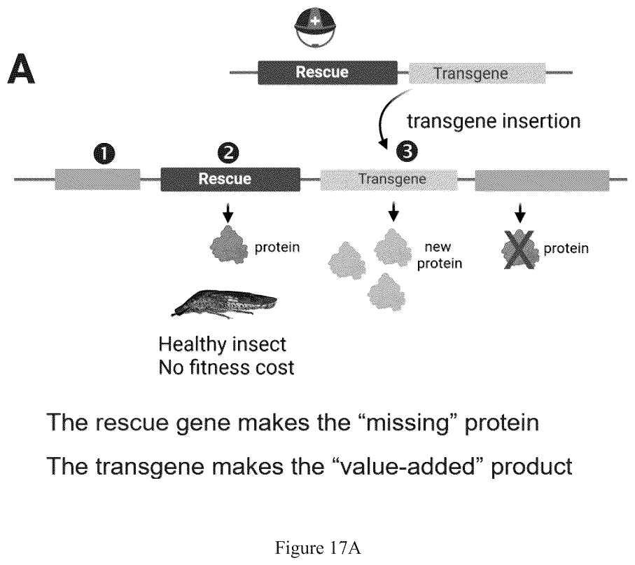

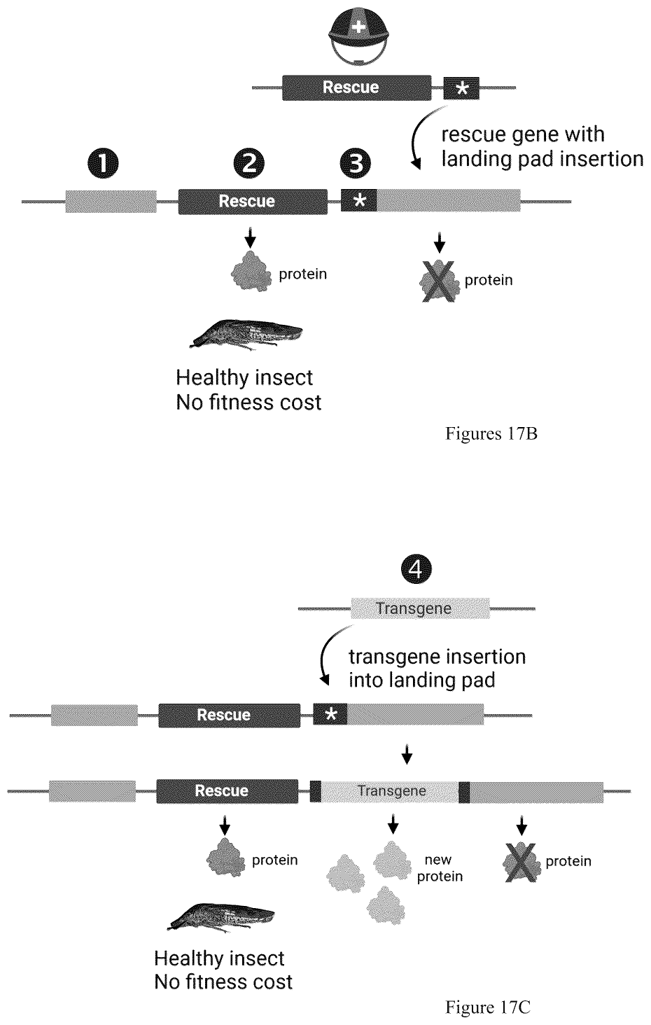

The ToD technology creates a synthetic GSH that could transform “any” gene into a GSH. Our strategy is simple. Since insertion of a cassette into a target gene causes loss of function, it often has a fitness cost (FIG. 8), we propose to complement the loss-of-function mutation with a chimeric rescue gene (e.g., see FIG. 17). We discuss the ToD concept in the context of insect gene drive. However, this technology is applicable to any organism so it could have wide ranging impact on mammalian, microorganism, plant and insect biotechnology. Our approach is based on gene complementation. The ability of a wild-type cDNA to substitute for the mutated gene. Described herein are exemplary methods to create a synthetic GSH that can transform “any” gene into a GSH (a target-on-demand, ToD).

The ToD technology breaks from the current dogma for GSH identification, which deliberately avoids insertional inactivation of a target gene due to potential fitness costs to an organism. Several other important features speak to the novelty of the ToD technology. While some genomics resources would be useful for the deployment of ToD technology in an organism, they are not essential. The ToD technology is not dependent on numerous deep and costly epi/genomics resources, the ability to propagate a species' cells in culture, access to large collections of insertional mutants, or large foot-print screens of mature transgenic organisms (FIG. 12, 13, 14).

The ToD strategy uses transcriptional units as the target sites for transgene integration. The minimal ToD gene cassette has a rescue gene and a landing site for the integration of one or more transgenes. Alternatively, a cargo-carrying ToD gene cassette includes a rescue gene and a transgene that encodes a value-added product. We restore function of the inactivated target gene by the integration of a ‘rescue’ gene that provides the target gene's product (FIG. 17). This functional complementation avoids any fitness costs to transgene inactivation. The rescue gene is simple in design. In the non-limiting example shown in FIG. 17, the rescue gene uses the target gene's promotor and its cDNA to assure that the target gene's protein is expressed at the correct time in development and in response to external cues. Therefore, ToD cassette's transgene resides in a transcriptionally active region chosen to confine expression of the transgene to the target tissue. The ToD technology is a fundamental shift from the conventional approach for GSH identification and has the advantage that potential GSH targets can be selected on the basis of the desired tissue-specific expression of the transgenes located in the gene cassette introduced into these GSH. Choosing target genes that have a desired developmental specificity or that are ubiquitously expressed should allow for the optimal epigenetic and genomic context to promote robust transgene expression; this should promote reliability, durability and efficacy of transgene expression.

Target genes (potential GSHs) can be identified by one of many strategies. Knowledge about orthologous genes in other species may help identify a target gene in a non-model organism. Alternatively, if RNA-seq data and a genome sequence (even at the scaffold level) are available, predicted expression of a target gene and its neighboring genes can be deduced to enable optimal target genes for the ToD strategy. As the ToD strategy is not based on robust genomics resources, testing a few (e.g., 5-6) target genes for their efficacy in a ToD strategy may assure that one or more GSHs are identified. It is noteworthy that even with robust genomics resources, multiple putative GSHs have been tested in most studies published to date.

In this Example, the deployment and development of the ToD technology in insects are further discussed, as GSHs are important for the successful deployment of sustainable gene drives. To date, genome insertion site targets for gene-drive cassettes have been genes with an easily identified morphological phenotypes, such as eye pigmentation or body color; in addition, genes critical for sex determination (e.g, doublesex) have been used in gene drive strategies in Anopheles gambiae (Kyrou et al. 2018) and Drosophila suzukii (Yadav et al. 2023). When used as target genes in mosquitoes, eye-color genes white (w) and cinnabar (cn) exhibit different gene drive efficiencies, 59% and 38%, respectively. Moreover, in our hands, w and cn mutations have a mild to severe fitness costs in Homalodisca vitripennis (glassy-winged sharp shooter, GWSS) and Bemisia tabaci (whitefly), respectively. For GWSS, disruption of cn interferes with paired matings but, luckily, not pool matings. GWSS w mutants have poor eclosion and slowed development. Despite these fitness costs, we have been able to maintain GWSS w and cn mutant colonies using pool matings for over 11 generations. Therefore, while cn is currently being used as a target gene for transgene insertion, cn is not optimal GSH for GWSS. In whitefly, w mutations are lethal. It is clear new target gene sites are needed. The ToD technology can solve these mild to severe fitness costs and provide optimal integration sites for transgene expression.

The ToD technology should enable robust and sustainable gene-drive strategies in insects as GSH loci that serve as optimal landing and launching pads for gene drive in insects are needed. Optimal target sites are also needed for the insertion of genes for sterile insect control programs and in transgenic strategies that would block pathogen transmission. There is substantial information indicating that the chromosomal integration sites for Cas9 and sgRNAs which are critical for many contemporary gene drives influences the success of gene drive strategy (López Del Amo et al. 2020).

Several simple criteria can be used to select a putative GSH (target gene) in a non-model organism with limited genomics resources. For example, a non-limiting, exemplary target gene may:
reside in a transcriptionally active region of the genome and, therefore, could have neighboring genes that are actively expressed. If epigenomics or DNase I data are available, open chromatin regions could be chosen.
be selected as a potential synthetic GSH site based on RNAseq data sets and other genomic/epigenomic data if these resources are available. But for many non-model organism, gene orthologs from other species can be selected for use and synteny with other organisms will allow prediction of neighboring genes.
be chosen based on the level of expression desired for the transgene. For example, the target gene should be expressed at a high level (if a high level of transgene expression is desired).
be expressed in the cell type, tissue of interest and with the developmental programming desired for transgene expression.
not use alternative splicing as a mechanism of gene regulation.
have a simple gene structure with few or no introns. This will limit the likelihood of regulatory elements residing within intronic regions or for alternate splicing to occur.
maybe a single-copy gene or a member of a multigene family that has gene-specific sgRNAs for use in CRISPR-mediated gene integration, and/or
should not be an essential gene. It is more likely that the complementation strategy will be successful for a gene that has only a small or modest fitness cost.

The transgene may confer a value-added trait to the organism. In testing the ToD technology, we will use a fluorescent reporter/marker gene to follow gene insertion events; this is important for organisms where CRISPR-mediate gene insertion occurs at low frequency. In using synthetic GSHs for interrogating biological processes or for biotechnology, the value-added trait includes traits beneficial to the organism or traits useful for pest insect control or traits useful for making product having industrial or therapeutic applications (e.g., product can be isolated or purified further). The transgene can use any native, alien or synthetic promoter, coding sequence, and 3′-flanking region. It would be advantageous to select a promoter to drive the transgene that is expressed in a similar manner to the target gene; but the target gene's promoter is not proposed to drive the transgene in certain embodiments, although it is possible one promoter could drive two genes with an IRES sequence or 2A peptide encoding sequence in between the two genes.

The rescue gene is constructed using knowledge of the target gene (the potential GSH). The rescue gene may utilize the target gene's promoter and 3′-flanking sequences to direct the expression of the target gene's protein in the correct cell types and tissue. The rescue gene's coding region could be the target gene's cDNA. If intronic sequences are important in modulating the expression level of the target gene, the rescue gene could include one or more introns that are known to be essential for driving native gene expression. However, this level of knowledge is not known for most genes in model or non-model organisms. For this reason, we focus on genes with simple structures. In addition, since complementation is achieved by using a single cDNA, it is important that alternative splicing of the target gene (if any) is not critical for its function.

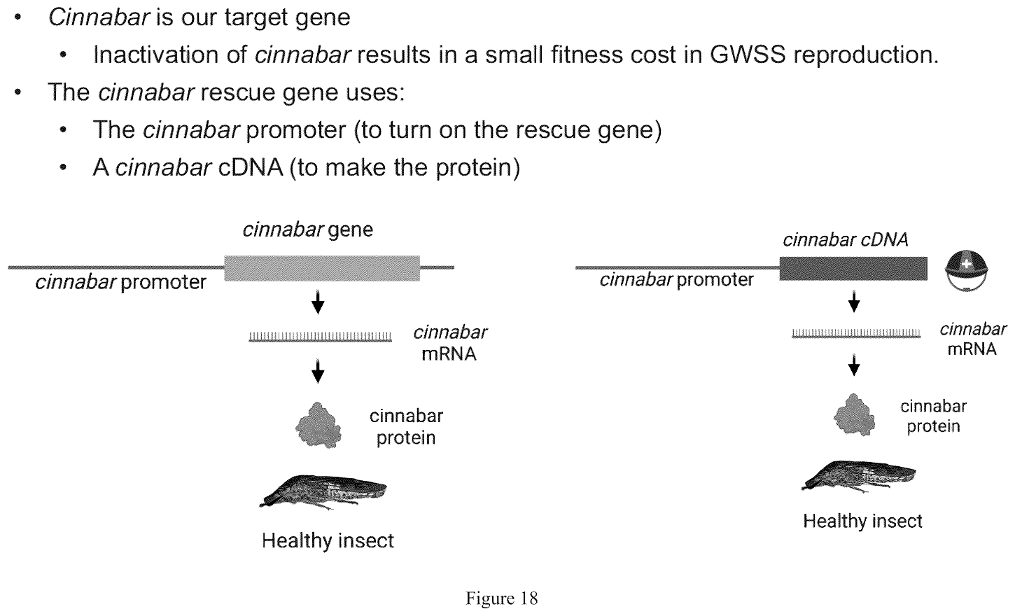

Two types of ToD constructs can be made. The minimal, receiving ToD cassette that harbors the rescue gene and landing pad (FIG. 17B). The exemplary landing pad contains a unique sgRNA cutting site. This can be used for CRISPR/Cas-mediated insertion of the transgene(s) to the receiving synthetic GSH. A cargo-loaded ToD can also be pursued (FIG. 17A). In this case, both the rescue gene and transgene residing within the ToD cassette are integrated into the target gene simultaneously. The minimal, receiving and cargo-loaded ToD cassettes can be assembled by a standard cloning method (e.g, Gibson assembly or GoldenGate technologies) or by synthesis of the gene cassette parts and assembly. For integration into the organism's genome, target gene homology arms could be included to promote HDR gene insertion. The homology arms may be dependent on the size of the ToD gene cassette; however, homology arms ranging from 800 to 1000 bp are typically used to precisely integrate genes by HDR into the organism's genome. FIG. 18 illustrates the concept of the target gene (putative GSH) and rescue gene. While we use the cn locus of GWSS, this concept is applicable to virtually any gene in any organism.

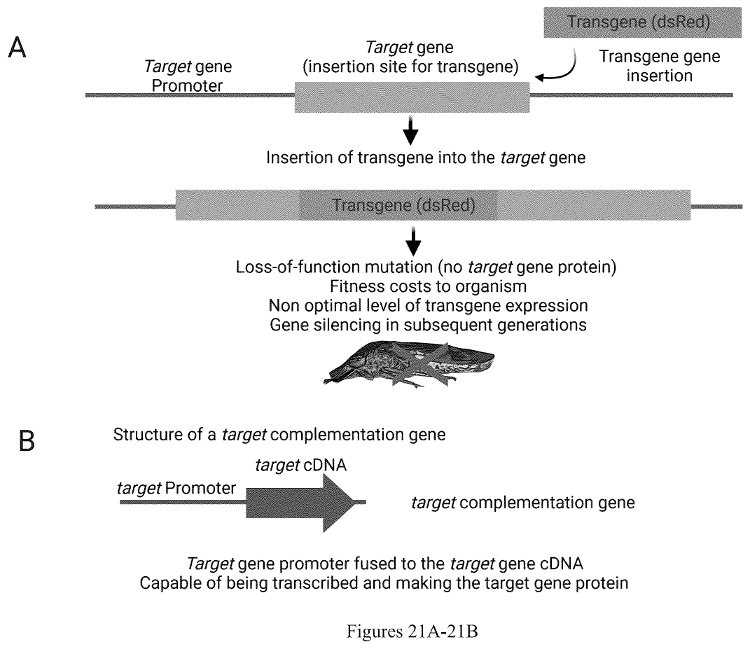

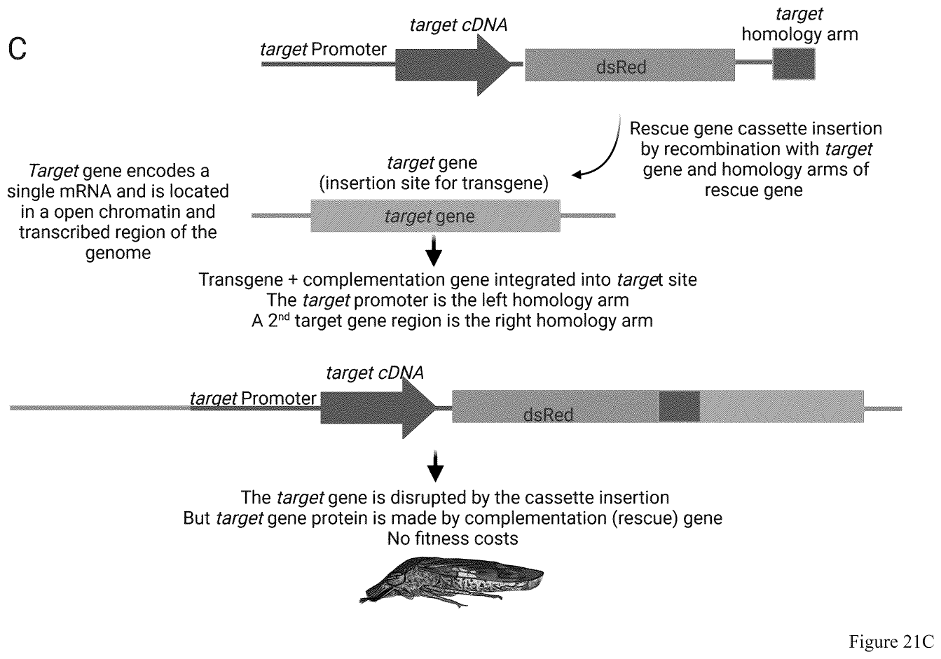

We illustrate the ToD scheme in FIG. 21. A target gene is the gene being tested as a synthetic GSH. When a gene cassette is inserted into a target gene, the target gene is inactivated causing mild to severe fitness costs (FIG. 21A). In the ToD gene cassette, we provide a chimeric rescue gene that complements the target gene deficiency caused by a cargo-loaded ToD cassette insertion (FIG. 21B). In this example of FIG. 21, the complementation (rescue) gene will be the target cDNA with its native promoter to promote accurate developmental and environmental expression of the rescue gene. The proof-of-concept ToD cassette (FIG. 21B) will also have a reporter gene (dsRed, the cargo) that produces a red fluorescent protein that allows us to follow cassette integration into the target by monitoring dsRed expression using fluorescence and mRNAs (qRT-PCR) and dsRed gene integration (PCR of genomic DNA). In this example of FIG. 21, we need homology arms for ToD cassette insertion into the target gene by Cas9 and sgRNAs via HDR or NHEJ. The promoter region will serve as the 5′-homology arm; this will bring all short and long 5′-regulatory regions in close proximity to the target cDNA. We will use ˜0.5 to 1-kb of the target gene as the right homology arm. This cargo-loaded ToD-rescue plasmid, sgRNA-cn and Cas9 will be introduced into the organism for ToD cassette gene integration by HDR or NHEJ (FIG. 21C).

Once a minimal, receiving synthetic GSH is identified, we can extend this technology for easy integration of other transgenes. For this application, a minimal, receiving ToD cassette is used (FIG. 17B). The first step is to integrate the rescue gene and a landing pad into the target gene. The landing pad is a unique sgRNA site that will allow precise integration of a transgene into this target gene location. The unique sgRNA can be identified and verified for lack of potential off-target sequences. Once a minimal ToD line is established it can be used for the insertion of any gene into the minimal synthetic GSH using the sgRNA, Cas endonuclease and a transgene sequence with homology arms.

### Proof of Concept Testing of the ToD Technology in Two Non-Model Insects

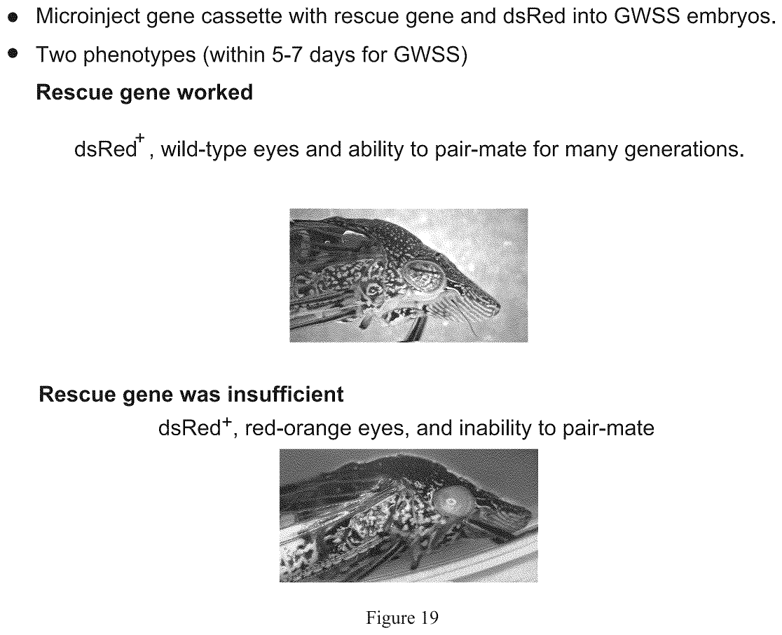

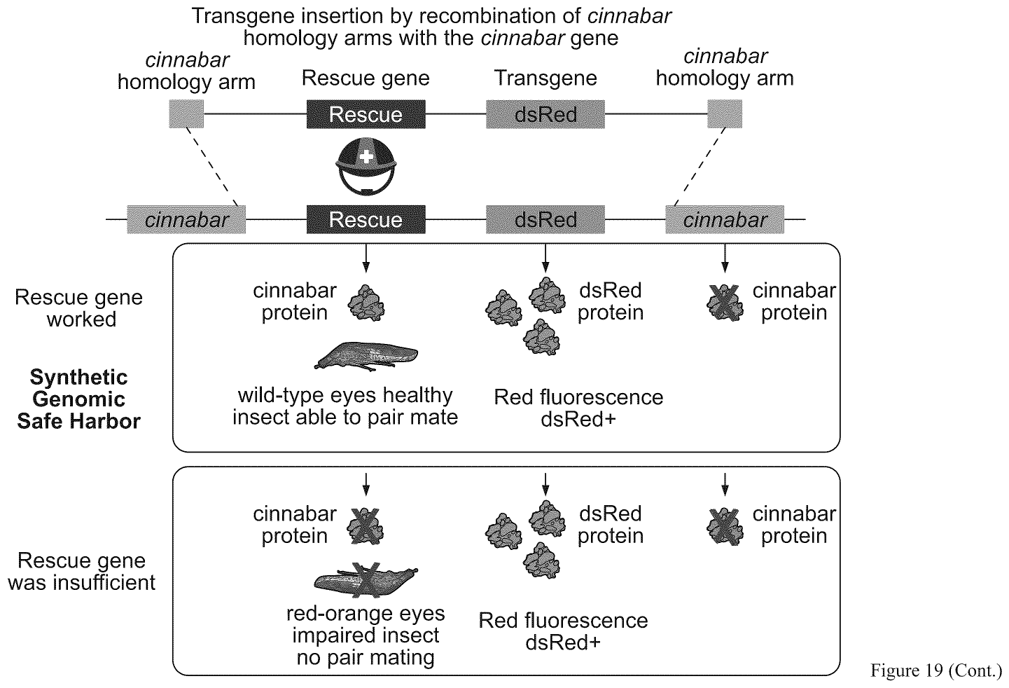

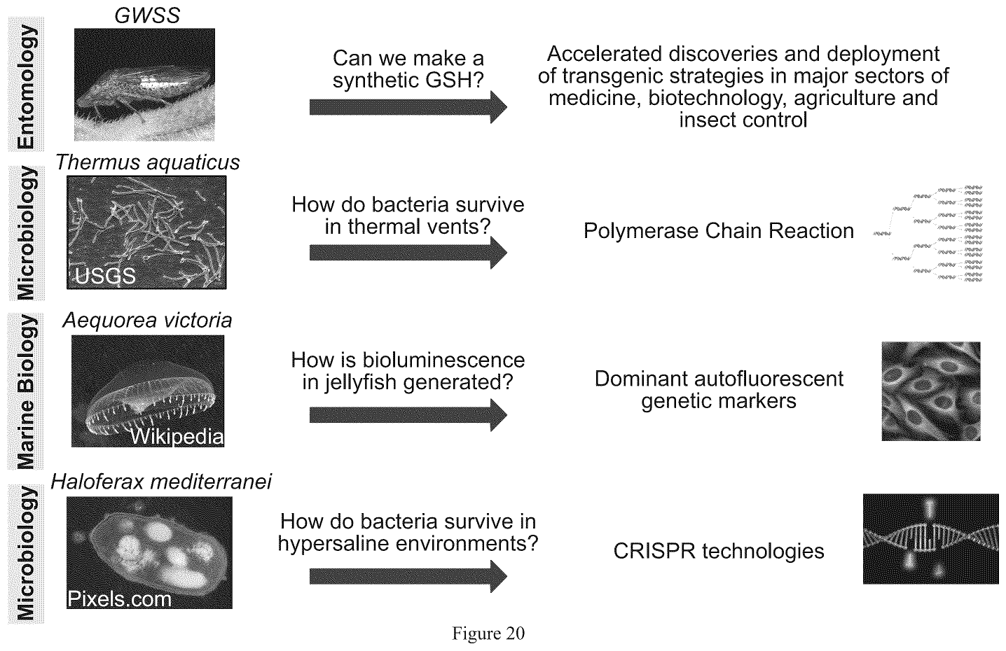

We test this ToD strategy using eye color genes in Homalodisca vitripennis (GWSS) and Bemisia tabaci (whitefly) due to our success with editing of these genes in these insects (de Souza Pacheco et al. 2022; Pacheco et al. 2022)(Atkinson and Walling, unpublished results). White (w) and cinnabar (cn) are used in GWSS and w and vermilion (v) are used with whiteflies. We know that the GWSS cn is not a GSH, as cn mutants have mild fitness costs that interfere with pair matings. We have shown that we can integrate genes into the GWSS cn with high efficiency using HDR and NHEJ technologies and we establish and maintain lines with pool matings (which bypasses the need for pair matings). Using the ToD strategy, four possible GO phenotypic classes could be generated: mosaic cn⁻ eyes, mosaic cn⁻ eyes and dsRED fluorescence, wild-type eyes, wild-type eyes and dsRED fluorescence (FIG. 19). Insects in each class will be pooled and virgin adults from this pool will be pair mated. Two phenotypes are indicative of the success or failure of the ToD strategy. G0 insects that have wild-type eyes (cn⁺) and are dsRed should be the result of complementation. Paired matings of these insects should have rates of egg hatch similar to wild-type insects. Whereas cn⁻/dsRed⁺ insects should yield no progeny from pair matings; they would represent an unsuccessful case of the ToD strategy. We also test the TOD strategy with the GWSS w and B. tabaci w genes as w mutants have more severe fitness costs in both of these organisms (Atkinson and Walling, unpublished results).

Impact: virtually “any” gene can be designed to be a synthetic GSH. The ToD strategy may challenge the dogma of avoiding insertion into transcriptionally active genes.

Further testing of optimal target genes for testing the synthetic GSH strategy will occur. To assure that the ToD strategy is easy to execute, the target genes could express a single RNA and be surrounded by transcriptionally active genes; these are simple criteria and the resources (even in non-model organisms) are often in place. We will have transcriptome data from seven GWSS organs that should allow selection of optimal target genes. A small number of target genes may need to be tested in each organism to provide the GSH site that promotes accurate and developmentally correct expression. This fast and efficient ToD method for GSH discovery could revolutionize gene-drive strategies in all organisms, having especially high impact on non-model organisms. If successful, this technology could potentially revolutionize biotechnology initiatives to express transgenes and gene drives in plants, animals and microbes.

### Proof-of-Concept Testing the ToD Technology in Insects—GWSS cn Gene

### Methods

1. We have identified the GWSS cn gene using two H. vitripennis genome sequences (Ettinger et al. 2021; Li et al. 2022). Unlike certain exemplary candidate target gene for synthetic GSHs that have a simple structure, the cn gene has 11 introns spanning 27,812 bp. Its first intron is very large (11,883).
2. We are determining the transcriptional start and stop sites of the cn gene using GWSS RNAs and the 5′- and 3′-RACE technology. This knowledge is used to accurately assess the boundaries of the first and last exon. The RACE strategy will allow us to determine if splice variants of the cn are used.
3. At the nucleotide level, we will have the 1-kb of the cn promoter and the cn˜1.6-kb cDNA synthesized in two segments (Twist) to allow Gibson assembly. The sizes of the promoter that serves as the left homology arm and the right homology could be tested to generate a high frequency of gene insertion. Currently, we know that short homology arms (e.g., about 100-200 nucleotides in length) facilitate oligonucleotide insertion into the cn gene. If needed, the rescue gene will be modified with alternate codons to allow discrimination of transcripts from the endogenous (inactivated) gene and the rescue gene.
4. The OpIE2:dsRed (reporter gene) will be PCR amplified with overlapping sequences to allow assembly with the cn rescue gene (step 3), the OpIE2:dsRed reporter gene, and the cn homology arm (˜1000 bp). The cargo-loaded ToD gene cassette comprises the rescue gene, reporter gene and right homology arm. In the plasmid vector, the ToD cargo-loaded cassette is flanked by unique sgRNA sites to facilitate plasmid linearization by Cas9 in embryos.
5. The ToD cassette plasmid, Cas9 protein (150-300 ng), and cn sgRNAs will be microinjected into GWSS embryos on sorghum leaves as described in de Souza Pacheco et al. (2022).
6. Microinjected embryos will be allowed to develop until day 5-6 on intact sorghum plants. At this time, embryos with surrounding sorghum leaf tissue are excised and placed on leaf disc medium described in Atkinson and Walling (2018).
7. The eye color of developing embryos can be assessed at day 5-6 to determine the frequency of orange and wild-type eyes. Orange-eyed GWSS will have genome site edits or will have ToD transgene insertion into the cn locus. Red-brown eyed GWSS (the wild-type phenotype) will be unedited GWSS or will be insects where the ToD construct has complemented the cn mutation caused by ToD transgene insertion.
8. When nymphs emerge, they will be separated into two phenotypic classes: orange eyes and red-brown eyes and raised by pooled mating.
9. A few days before the GWSSs reach their 3rd instar, insects will be confined individually to plants or to rooted leaf discs in vitro (Atkinson and Walling 2018). Insects will be allowed to molt and exuviae will be collected. Insects will remain in isolation until their genotypes are determined.
10. DNA from each exuvia will be extracted. The presence of the ToD cassette in the genome will be determine using PCR using rescue gene and dsRed gene-specific primers.
11. Genotyped insects will be used to make four colonies—class 1 to 4 (Table 1). Class 1 indicated that the rescue strategy worked.

TABLE 1
Phenotypic and genotypic classes of GWSS

| | dsRed | | Rescue | |
|---|---|---|---|---|
| | gene | Red | gene | |
| Phenotype Class | (PCR) | Fluorescence | (PCR) | Eye color |
| Class 1: | positive | dsRed | positive | wildtype |
| ToD successful | | Fluorescence | | |
| Class 2: | negative | negative | negative | wildtype |
| Non-edited insects or | | | | |
| mutants with no | | | | |
| phenotype | | | | |
| Class 3: | negative | negative | negative | orange |
| cn edited | | | | |
| Class 4: | positive | dsRed | positive | orange |
| No complementation | | Fluorescence | | |
| from ToD | | | | |

12. Insects in the four colonies will be grown to maturity. Insects from class 1 and class 3 will be further characterized as they assess the efficiency of the ToD strategy.
13. Fecund females will be mated with several males from the same colony.
14. Fertilized females will deposit eggs on sorghum leaves. Progeny from each G0 mother (G1 insects) will be used to form a colony.
15. Phenotypes and genotypes of G1 insects will be assessed as described above. Expression of the inactivated cn gene, cn rescue gene, and dsRed reporter gene will be assessed by qRT-PCR.
16. The frequency of class 1 insects will reflect the efficiency of the ToD technology.
17. Stable inheritance and expression of the rescue gene and dsRed transgene will be determined.

### Constructing the Minimal ToD Cassette to Easily Deploy the ToD Technology

We will make the simpler minimal ToD cassette that will insert the rescue gene and a landing pad into the GWSS cn gene, or another target gene (an optimal target gene for sGSH). The landing pad will have one or multiple unique sgRNA cutting sites for insertion of transgene(s). Due to the high efficiency of HDR gene insertion in GWSS, we can avoid the use of a reporter gene in this construct and directly screen for gene insertion events by PCR.
1. The complementation sequence (promoter, rescue gene cDNA, 3′-flanking region) and a downstream synthetic landing pad will be synthesized and assembled.
2. In this example, the exemplary 500-bp landing pad region will not have homology to the cn gene. Furthermore, a unique sgRNA sequence with a PAM will be included in this landing pad region.
3. If multiple transgenes need to be inserted into the minimal synthetic GSH sequentially, we can include multiple unique sgRNAs each separated by approximately 100 bp (filler sequence).
4. The landing pad sequence will be fused to a 3′ homology arm using downstream portion of the cn gene (same homology arm as in the cargo-loaded ToD construct). This region will be synthesized and assembled with the complementation sequence. The minimal ToD cassette is flanked by unique sgRNA sites on a plasmid vector to facilitate plasmid linearization by Cas9 in embryos.
5. HDR will be used to insert the minimal ToD cassette into the cn gene as described above for the cargo-loaded ToD (Steps 5-6).
6. The eye color of developing embryos can be assessed at day 5-6 to determine the frequency of orange and wild-type eyes. Orange-eyed GWSS will have genome site edits or will have ToD transgene insertion into the cn locus. Red-brown eyed GWSS (the wild-type phenotype) will be unedited GWSS or will be insects where the ToD construct has complemented the cn mutation caused by ToD transgene insertion.
7. When nymphs emerge, they will be separated into two phenotypic classes: orange eyes and red-brown eyes and raised by pooled mating.
8. A few days before the GWSSs reach their 3rd instar, insects will be confined individually to plants or to rooted leaf discs in vitro (Atkinson and Walling 2018). Insects will be allowed to molt and exuviae will be collected. Insects will remain in isolation until their genotypes are determined.
9. DNA from each exuvia will be extracted. The presence of the minimal ToD cassette in the genome will be determined using PCR using rescue gene and dsRed gene-specific primers.
10. Genotyped insects will be used to make four colonies—class 1 to 4 (Table 1). Class 1 indicated that the rescue strategy worked.
11. The minimal ToD line will be sequenced verified across the ToD cassette insertion region.
12. One or multiple transgenes can be inserted into a single sgRNA site that resides in the landing pad. Transgenes will have 5′ and 3′ homology arms to allow integration into a landing site. Construction will proceed as described above. We will genotype each insect as described above to identify insects carrying both the synthetic GSH and the target gene.
13. Target gene expression will be assessed by qRT-PCR and any other relevant technology to measure protein levels and or metabolite levels.

### Second Generation Synthetic GSHs in GWSS—Optimal GSHs for Constitutive Target Gene Expression

Optimal synthetic GSHs will have a simple gene structure with few or no introns, be surrounded by actively transcribed genes in the genome, and be constitutively expressed. With the limited genomics resources at hand for GWSS, we will identify such candidate genes.
1. We are identifying constitutively expressed genes using our ovary, testes, salivary gland and cibarium/precibarium transcriptomes. In addition, malphigian tube, wing. leg, abdomen, eye, and whole male and female transcriptomes are available. We are identifying genes that are highly and moderately expressed genes in all samples. We are organizing genes based on their chromosomal or scaffold location and determine if candidate target genes are surrounded by genes that are also constitutively expressed.
2. Our ovary, testes, salivary gland and cibarium/precibarium transcriptomes will allow us to identify constitutively expressed genes that make a single transcript (e.g., no alternate splicing).
3. Mapping to the GWSS chromosomal assembly will indicate the number of exons/introns.
4. We will select target genes for further characterization as synthetic GSHs based on their transcripts being detected in all samples examined, their level, absence of alternative splicing, and simple gene structure. In addition, the genes in the target gene region should have a similar gene expression profile.
5. Genes can be single copy or members of small gene families.
6. We will test these target genes for their efficacy as synthetic GSHs using the methods describe for GWSS cn. We will test a cargo-loaded ToD construct first. If promising, we will construct the minimal ToD construct for testing of other transgenes.

### Assessing the ToD Technology in Insects—GWSS Using the White Gene

The methods being used are similar to the GWSS cn gene. The w gene cargo-loaded ToD construct with be the 2^{nd} proof-of-concept experiment due to the ease of GWSS editing. The w ToD construct will use the w promoter, w cDNA and w homology arm. The reporter gene and its promoter will be the OpIE2:dsRed construct. A minimal ToD cassette will also be assembled and tested for use for integrating transgenes as described for the cn minimal ToD cassette.

### Assessing the ToD Technology in Insects—Bemisia tabaci Using the Vermilion and White Genes

The methods being used to construct the vermilion (v) and w ToD constructs will be similar to the GWSS cn gene ToD. The two B. tabaci genes will be the 3^{rd} and 4^{th} proof-of-concept experiments for the ToD technology. The w ToD construct comprising w rescue gene will include the w promoter, w cDNA and a w homology arm. The v ToD construct comprising w rescue gene will include the v promoter, v cDNA and v homology arm. The transgene (reporter gene and its promoter) will be the OpIE2:dsRed construct. The rescue gene and transgene will be assembled to form the cargo-loaded ToD cassette. The methods for introducing Cas9, sgRNAs, and plasmids into B. tabaci embryos are described in US patent application publication No. US 20210105986 (Atkinson and Walling 2018), which is incorporated by reference herein.

Whiteflies will be assessed for phenotypes (eye-color, mortality, dsRed fluorescence) to assess the utility of the rescue genes in this insect. Minimal ToD cassettes will be assembled and tested as described for GWSS.

There is comparatively limited organ-specific transcriptome data for B. tabaci. We have salivary gland and abdomen, as well as whole insect and virus-infected transcriptomes to use for identification of transgenes that are constitutively expressed. The steps for identifying and testing candidate target genes as synthetic GSHs will follow the protocols described above.

### Assessing the ToD Technology in Plants

The ToD technology would have a large impact on crop biotechnology and plant cell cultures used for bioreactor production of macromolecules, as well as the study of model plants such as Arabidopsis thaliana. The criteria for a GSH for transgene expression in intact plant vs plants cells grown in bioreactors may be different. Many genes essential for plant development and growth are not needed in plant cell culture; there are marked distinctions in intact plants versus immortalized plant cell culture transcriptomes (Tanurdzic et al. 2008; Iwase et al. 2005). The GSHs for transgenes used in plant cell culture-based biotechnologies would emphasize high transgene expression with high yields of recombinant proteins or metabolites (Rozov et al. 2022). Rozov et al (2022) inserted a modified human interferon gene into a transcriptionally active region upstream of a Histone3 gene that is expressed constitutively during prophase. Protein yields were 2-5 fold more than random transgenic insertion events. In addition, large gene cassettes have also been inserted into a region adjacent to a constitutively expressed ubiquitin gene by Cre-lox technologies and both regulated and constitutive promoters were accurately used (Pathak and Srivastava 2020).

The proof-of-concept experiments are proposed for rice. CRISPR/Cas-mediated integration of a 5.2-kb carotenoid biosynthesis construct into two GSHs of rice has been successful (Dong et al. 2020).

### Methods

1. We will use phytoene desaturase (aka, phytoene synthase, PSY) as a target gene. Rice has three PSY genes; PSY1 and PSY2 are light activated and PSY3 is stress regulated. Inactivation of rice PSY genes by RNAi gives a distinct bleaching phenotype in photosynthetically active organs (Miki and Shimamoto 2004).
2. We will also identify candidate target genes for use in intact plants and in plant cell culture. Given the success of Rozov et al (2022) in transcriptionally active regions, we will identify gene families that are constitutively expressed in rice. A gene that is located between other actively transcribed genes will be selected as a target gene. The target gene must have gene-specific sgRNA sites. In certain embodiments, the target gene should not use alternative splicing for gene regulation.
3. For each gene tested as a synthetic GSH, complementation sequence will be constructed using the principles for the insect rescue genes described above. For each target gene tested, a ˜1000-bp promoter and cDNA will be synthesized and assembled. The complementation sequence will be then be assembled with 35S:eGFP, which is a good reporter gene in plant cells. The ˜1-kb target gene promoter will be used as the left homology arm and a downstream region of target gene will be used as the right homology arm. Dong et al. (2020) used 500-800 bp homology arms to facilitate HDR. However, they showed that gene integration primarily occurred by NHEJ in their experiment.
4. The ToD cassette will be cloned into the donor plasmid (pAccB). sgRNA-PSY will flank the ToD cassette to release the cassette from its plasmid vector. The CRISPR plasmid pCam1300-CRIPS-B will be modified (Dong et al. 2020). This plasmid will express Cas9 and the U6:sgRNA-PSY. The sgRNA cuts the endogenous PSY gene in the rice genome and the two cut sites on the pAccB-ToD plasmid to release the ToD cassette.
5. Plasmids will be delivered by particle bombardment into rice calli as described by Dong et al. (2020). Transgenic calli expressing the CRISPR plasmid will be selected and regenerated into seedlings. Seedlings will be phenotyped and genotyped. Several phenotypes are expected as outlined in Table 1. Class 1 plants are reflective of the success of the ToD technology. As outlined in Dong et al (2020), the presence or absence of the CRISPR plasmid will also be determined in the Class 1 and 2 plants.
6. Once a cargo-loaded ToD is verified as a synthetic GSH, we will construct rice that has a minimal ToD with a rescue gene and a landing pad for introduction of transgene(s) for plant improvement and biotechnology.

TABLE 2
Phenotypic and genotypic classes of rice seedlings

| | eGFP | | Rescue | Plants |
|---|---|---|---|---|
| | gene | GFP | gene | (green or |
| Phenotype Class | (PCR) | Fluorescence | (PCR) | bleached) |
| Class 1: | positive | GFP | positive | Green |
| ToD was successful | | Fluorescence | | |
| Class 2: | positive | GFP | positive | bleached |
| Unsuccessful ToD | | Fluorescence | | |
| Class 3: | negative | negative | negative | bleached |
| PSY gene was edited | | | | |
| Class 4: | negative | negative | negative | negative |
| untransformed plant | | | | |

### Assessing the ToD Technology in Mammalian (Human) Cell Culture

### Methods

1. Current human GSHs are not within genes and are not useful to test the ToD technology.
2. Human candidate GSH (target genes) will be selected using existing transcriptomes and the abundance of genomics and epigenomic resources. We will leverage these studies and identify actively transcribed genes meet criteria 3 to 8 (see above) (Dabiri et al. 2023; Papapetrou and Schambach 2016b). The most important criterion for humans is avoidance of regions that are >300 kb from known cancer-associated genes that are in transcriptionally active regions (Papapetrou et al. 2011).
3. Alternative splicing is extensively understood in humans and the majority of protein diversity in humans is due to alternative splicing (Jiang and Chen 2021). For this reason, in certain embodiments we will choose genes that are not alternatively spliced, as preferably only one gene product will be made by the ToD rescue gene.
4. For each synthetic GSH tested in the ToD technology, rescue genes will be constructed using the principles for the insect rescue genes described above. For each target gene tested, in certain embodiments, a ˜1000-bp promoter and cDNA will be synthesized and assembled. The complementation sequence will be then be assembled with the mCherry (or eGFP) reporter that is documented to be expressed in human iPS cells. Target gene-reporter gene fusions can also be tested. A ˜1-kb target gene promoter will be used as the left homology arm and a downstream region of target gene will be used as the right homology arm.
5. An appropriate cell cells or iPS cells that exhibit characteristic human embryonic stem (hES) cell morphology (Papapetrou et al. 2011) will be used to integrate ToD cassettes using established for gene integration using Cas endonucleases and sgRNAs.
6. ToD cassette-expressing cell lines will be will be identified by cell sorting as described by Papapetrou et al. (2011). mCherry/eGFP lines will be established and compared to non-transgenic cell lines. eGFP positive cells will be assessed for the rescue gene and endogenous gene RNAs (RNAs from downstream exons), e.g., using qRT-PCR.
7. Cells lines will be carried for several generations and the frequency of rescue and mCherry eGFP reporter gene silencing will be assessed using FACs cell sorting and qRT-PCR.
8. Stable high level expression of the mCherry reporter gene and rescue gene and lack of expression of the downstream exons of the target gene will indicate that the ToD technology is feasible.
9. Once a cargo-loaded ToD is verified as a synthetic GSH, we will construct cell line that has a minimal ToD with a rescue gene and a landing pad for introduction of transgene(s) for cell therapies and biotechnology.

### Assessing the ToD Technology in Mice

Detailed surgical procedures required for vasectomies, removing embryos from pregnant, euthanized mice, microinjection of embryos in vitro, incubation of embryos in vitro, and subsequent insertion of these embryos into the ampulla of the oviduct via the infundibulum in anesthetized females can be found in Bunting et al. (2022). The outcome of the experiments described in this paper are mice that have been gene edited, these being confirmed both by phenotype and by DNA sequencing of PCR products generated from the target site of the gene editing. To test our ToD approach in these mice, additions or modifications to this protocol are as follows:
1. Identify a target gene using the criteria described above.
2. Identify the promoter region, the start point of transcription, the transcriptional map of the coding region and any possible 3′ regulatory elements.
3. Construct the exogenous fusion sequence to include the promoter sequence, cDNA sequence, and any 3′ regulatory sequence flanking the cDNA of this target gene and also a fluorescent protein-encoding gene as transgene under the control of a constitutive promoter.
4. A plasmid containing this cassette is injected, with Cas9 protein and an sgRNA specific to the target into early mouse embryos, which are then implanted into surrogate mothers.
5. Adult mice are assessed for the presence of the fluorescent genetic marker and the absence of a mutant phenotype that would arise from the inactivation of the ToD target. These mice are used to establish homozygous lines which are then monitored for genetic fitness using standard parameters and compared with a genetic line (if it can be created) of mice that have the mutant phenotype expected from the inactivation of the transgene and exhibit fluorescence. Sequencing across the target site will confirm genotype using genomic DNA prepared from mouse tails.
6. Once a cargo-loaded ToD is verified as a synthetic GSH, we will also construct mouse line that has a minimal ToD with a rescue gene and a landing pad for introduction of transgene(s) for cell therapies and biotechnology.

### References in Example 2

Arras S.D., Chitty J.L., Blake K.L., Schulz B.L., and Fraser J.A. (2015) A genomic safe haven for mutant complementation in Cryptococcus neoformans. PLOS One, 10, e0122916. doi:10.1371/journal.pone.0122916
Asad M., Liu D., Li J., Chen J., and Yang G. (2022) Development of CRISPR/Cas9-Mediated Gene-Drive Construct Targeting the Phenotypic Gene in Plutella xylostella. Frontiers in Physiology, 13. doi:10.3389/fphys.2022.938621
Atkinson P.A., and Walling L.L. (2018) Method for Genetic Manipulation of Sap-feeding Insects. US patent application publication number US 20210105986
Autio M.I., Motakis E., Perrin A., Bin Amin T., Tiang Z., Do D.V., Wang J., Tan J., Tan W.X., Ding S., Teo A.K.K., and Foo R.S.Y. (2021) Computationally defined human genomic safe harbour loci validated in vitro for stable transgene expression. Human Gene Therapy, 32, A67-A68
Aznauryan E., Yermanos A., Kinzina E., Devaux A., Kapetanovic E., Milanova D., Church G.M., and Reddy S.T. (2022) Discovery and validation of human genomic safe harbor sites for gene and cell therapies. Cell Reports Methods, 2, 100154. doi:doi.org/10.1016/j.crmeth.2021.100154
Balmas E., Sozza F., Bottini S., Ratto M.L., Savore G., Becca S., Snijders K.E., and Bertero A. (2023) Manipulating and studying gene function in human pluripotent stem cell models. FEBS Lett. doi:10.1002/1873-3468.14709
Bunting M.D., Pfitzner C., Gierus L., White M., Piltz S., and Thomas P.Q. (2022) Generation of Gene Drive Mice for Invasive Pest Population Suppression. Methods Mol Biol, 2495, 203-230. doi:10.1007/978-1-0716-2301-5_11
Chekulaeva M., and Filipowicz W. (2009) Mechanisms of miRNA-mediated post-transcriptional regulation in animal cells. Curr Opin Cell Biol, 21, 452-460. doi:10.1016/j.ceb.2009.04.009
Dabiri H., Safarzadeh Kozani P., Habibi Anbouhi M., Mirzaee Godarzee M., Haddadi M.H., Basiri M., Ziaei V., Sadeghizadeh M., and Hajizadeh Saffar E. (2023) Site-specific transgene integration in chimeric antigen receptor (CAR) T cell therapies. Biomark Res, 11, 67. doi:10.1186/s40364-023-00509-1
de Souza Pacheco I., Doss A .-L.A., Vindiola B.G., Brown D.J., Ettinger C.L., Stajich J.E., Redak R.A., Walling L.L., and Atkinson P.W. (2022) Efficient CRISPR/Cas9-mediated genome modification of the glassy-winged sharpshooter Homalodisca vitripennis (Germar). Scientific Reports, 12. doi:s
Dong O.X.O., Yu S., Jain R., Zhang N., Duong P.Q., Butler C., Li Y., Lipzen A., Martin J.A., Barry K.W., Schmutz J., Tian L., and Ronald P.C. (2020) Marker-free carotenoid-enriched rice generated through targeted gene insertion using CRISPR-Cas9. Nature Communications, 11. doi:10.1038/s41467-020-14981-y
Ettinger C.L., Bryne F.J., Collin M.A., Carter-House D., Walling L.L., Atkinson P.W., Redak R.A., and Stajich J.E. (2021) Improved draft reference genome for the Glassy-winged Sharpshooter (Homalodisca vitripennis), a vector for Pierce's disease. G3-Genome Report, October 2021, jkab255, doi.org/10.1093/g3journal/jkab255,
Furukawa T., van Rhijn N., Chown H., Rhodes J., Alfuraiji N., Fortune-Grant R., Bignell E., Fisher M.C., and Bromley M. (2022) Exploring a novel genomic safe-haven site in the human pathogenic mould Aspergillus fumigatus. Fungal Genet Biol, 161, 103702. doi:10.1016/j.fgb.2022.103702
Ittiprasert W., Moescheid M.F., Chaparro C., Mann V.H., Quack T., Rodpai R., Miller A., Wisitpongpun P., Buakaew W., Mentink-Kane M., Schmid S., Popratiloff A., Grevelding C.G., Grunau C., and Brindley P.J. (2023) Targeted insertion and reporter transgene activity at a gene safe harbor of the human blood fluke, Schistosoma mansoni. Cell Rep Methods, 3, 100535. doi:10.1016/j.crmeth.2023.100535
Iwase A., Ishii H., Aoyagi H., Ohme-Takagi M., and Tanaka H. (2005) Comparative analyses of the gene expression profiles of Arabidopsis intact plant and cultured cells. Biotechnol Lett, 27, 1097-1103. doi:10.1007/s10529-005-8456-x
Jeong B.R., Jang J., and Jin E. (2023) Genome engineering via gene editing technologies in microalgae. Bioresour Technol, 373, 128701. doi:10.1016/j.biortech.2023.128701
Jiang W., and Chen L. (2021) Alternative splicing: Human disease and quantitative analysis from high-throughput sequencing. Comput Struct Biotechnol J, 19, 183-195. doi:10.1016/j.csbj.2020.12.009
Jung K.-H., An G., and Ronald P.C. (2008) Towards a better bowl of rice: assigning function to tens of thousands of rice genes. Nature Reviews Genetics, 9, 91-101. doi:10.1038/nrg2286
Kyrou K., Hammond A.M., Galizi R., Kranjc N., Burt A., Beaghton A.K., Nolan T., and Crisanti A. (2018) A CRISPR-Cas9 gene drive targeting doublesex causes complete population suppression in caged Anopheles gambiae mosquitoes. Nature Biotechnology, 36, 1062-1066. doi:10.1038/nbt.4245
Li G., Jain R., Chern M., Pham N.T., Martin J.A., Wei T., Schackwitz W.S., Lipzen A.M., Duong P.Q., Jones K.C., Jiang L., Ruan D., Bauer D., Peng Y., Barry K.W., Schmutz J., and Ronald P.C. (2017) The Sequences of 1504 Mutants in the Model Rice Variety Kitaake Facilitate Rapid Functional Genomic Studies. The Plant Cell, 29, 1218-1231. doi:10.1105/tpc. 17.00154
Li Z., Li Y., Xue A.Z., Dang V., Holmes V.R., Johnston J.S., Barrick J.E., and Moran N.A. (2022) The Genomic Basis of Evolutionary Novelties in a Leafhopper. Molecular Biology and Evolution, 39. doi:10.1093/molbev/msac184
López Del Amo V., Bishop A.L., Sánchez C H.M., Bennett J.B., Feng X., Marshall J.M., Bier E., and Gantz V.M. (2020) A transcomplementing gene drive provides a flexible platform for laboratory investigation and potential field deployment. Nature Communications, 11, 352. doi:10.1038/s41467-019-13977-7
Ma X., Zeng W.J., Wang L., Cheng R., Zhao Z. Y., Huang C.Y., Sun Z.X., Tao P.P., Wang T., Zhang J.F., Liu L., Duan X., and Niu D. (2022) Validation of reliable safe harbor locus for efficient porcine transgenesis. Functional & Integrative Genomics. doi:10.1007/s10142-022-00859-3
Malaiwong N., Porta-de-la-Riva M., and Krieg M. (2023) FLInt: single shot safe harbor transgene integration via Fluorescent Landmark Interference. G3 (Bethesda), 13. doi:10.1093/g3journal/jkad041
Miki D., and Shimamoto K. (2004) Simple RNAi Vectors for Stable and Transient Suppression of Gene Function in Rice. Plant and Cell Physiology, 45, 490-495. doi:10.1093/pcp/pch048
Miyata Y., Tokumoto S., Arai T., Shaikhutdinov N., Deviatiiarov R., Fuse H., Gogoleva N., Garushyants S., Cherkasov A., Ryabova A., Gazizova G., Cornette R., Shagimardanova E., Gusev O., and Kikawada T. (2022a) Identification of Genomic Safe Harbors in the Anhydrobiotic Cell Line, Pv11. Genes, 13. doi:10.3390/genes13030406
Miyata Y., Tokumoto S., Arai T., Shaikhutdinov N., Deviatiiarov R., Fuse H., Gogoleva N., Garushyants S., Cherkasov A., Ryabova A., Gazizova G., Cornette R., Shagimardanova E., Gusev O., and Kikawada T. (2022b) Identification of Genomic Safe Harbors in the Anhydrobiotic Cell Line, Pv11. Genes, 13, 406. doi.org/410.3390/genes13030406. doi:10.3390/genes13030406
Odak A., Yuan H., Feucht J., Mansilla-Soto J., Eyquem J., Leslie C., and Sadelain M. (2020) Targeted Integration of a CAR at a Novel Genomic Safe Harbor Directs Potent Therapeutic Outcomes. Blood, 136. doi:10.1182/blood-2020-141967
Pacheco I.D., Walling L.L., and Atkinson P.W. (2022) Gene Editing and Genetic Control of Hemipteran Pests: Progress, Challenges and Perspectives. Front Bioeng Biotechnol, 10, 900785. doi:10.3389/fbioe.2022.900785
Papapetrou E.P., Lee G., Malani N., Setty M., Riviere I., Tirunagari L.M.S., Kadota K., Roth S.L., Giardina P., Viale A., Leslie C., Bushman F.D., Studer L., and Sadelain M. (2011) Genomic safe harbors permit high β-globin transgene expression in thalassemia induced pluripotent stem cells. Nature Biotechnology, 29, 73-78. doi:10.1038/nbt.1717
Papapetrou E.P., and Schambach A. (2016a) Gene Insertion Into Genomic Safe Harbors for Human Gene Therapy. Mol Ther, 24, 678-684. doi:10.1038/mt.2016.38
Papapetrou E.P., and Schambach A. (2016b) Gene Insertion Into Genomic Safe Harbors for Human Gene Therapy. Molecular Therapy, 24, 678-684. doi:10.1038/mt.2016.38
Pathak B., and Srivastava V. (2020) Recombinase-mediated integration of a multigene cassette in rice leads to stable expression and inheritance of the stacked locus. Plant Direct, 4, e00236. doi:10.1002/pld3.236
Rozov S.M., Permyakova N.V., Sidorchuk Y.V., and Deineko E.V. (2022) Optimization of Genome Knock-In Method: Search for the Most Efficient Genome Regions for Transgene Expression in Plants. International Journal of Molecular Sciences, 23. doi:10.3390/ijms23084416
Shibata Y., Okumura A., Mochii M., and Suzuki K.T. (2023) Protocols for transgenesis at a safe harbor site in the Xenopus laevis genome using CRISPR-Cas9. STAR Protoc, 4, 102382. doi:10.1016/j.xpro.2023.102382
Shibata Y., Suzuki M., Hirose N., Takayama A., Sanbo C., Inoue T., Umesono Y., Agata K., Ueno N., Suzuki K .-i.T., and Mochii M. (2022) CRISPR/Cas9-based simple transgenesis in Xenopus laevis. Dev Biol, 489, 76-83. doi:doi.org/10.1016/j.ydbio.2022.06.001
Tanurdzic M., Vaughn M.W., Jiang H., Lee T.J., Slotkin R.K., Sosinski B., Thompson W.F., Doerge R.W., and Martienssen R.A. (2008) Epigenomic consequences of immortalized plant cell suspension culture. PLOS Biol, 6, 2880-2895. doi:10.1371/journal.pbio.0060302
Van Meter E.N., Onyango J.A., and Teske K.A. (2020) A review of currently identified small molecule modulators of microRNA function. Eur J Med Chem, 188, 112008. doi:10.1016/j.ejmech.2019.112008
Xu X., Harvey-Samuel T., Siddiqui H.A., De Ang J.X., Anderson M.E., Reitmayer C.M., Lovett E., Leftwich P.T., You M., and Alphey L. (2022) Toward a CRISPR-Cas9-based gene drive in the diamondback moth Plutella xylostella. The CRISPR Journal, 5, 224-236. doi:10.1089/crispr.2021.0129
Yadav A.K., Butler C., Yamamoto A., Patil A.A., Lloyd A.L., and Scott M.J. (2023) CRISPR/Cas9-based split homing gene drive targeting doublesex for population suppression of the global fruit pest Drosophila suzukii. Proc Natl Acad Sci USA, 120, e2301525120. doi:10.1073/pnas.2301525120
Yamamoto Y., and Gerbi S.A. (2018) Making ends meet: targeted integration of DNA fragments by genome editing. Chromosoma, 127, 405-420. doi:10.1007/s00412-018-0677-6

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A synthetic genomic safe harbor (GSH) in a genome, the synthetic GSH comprising an exogenous fusion sequence that is inserted at the locus of an endogenous target gene of the genome, wherein the fusion sequence comprises:
(a) a landing sequence comprising at least one cutting sequence, or a transgene sequence encoding a transgene product, and
(b) a complementation sequence comprising a rescue gene sequence that encodes a target gene product.

2. The synthetic GSH of claim 1, wherein the fusion sequence comprises the landing sequence.

3. The synthetic GSH of claim 1, wherein the fusion sequence comprises the transgene sequence, and optionally, further comprises a promoter sequence for the transgene sequence.

4. The synthetic GSH of claim 2, wherein the cutting sequence comprises a PAM sequence and a gRNA related sequence; and/or wherein the landing sequence comprises two or more unique cutting sequences.

5. (canceled)

6. The synthetic GSH of claim 1, wherein the complementation sequence further comprises a promoter sequence for the rescue gene sequence.

7. (canceled)

8. The synthetic GSH of claim 1, wherein the rescue gene sequence comprises a cDNA sequence that does not comprise an altered codon(s) relative to the native encoding sequence of the endogenous target gene.

9-16. (canceled)

17. The synthetic GSH of claim 1, wherein the fusion sequence further comprises an internal ribosomal entry site (IRES) sequence or a 2A peptide encoding sequence placed between the complementation sequence, and the transgene sequence or the landing sequence; and/or wherein the fusion sequence further comprises a flanking sequence that is homologous to sequence at the locus of the endogenous target gene.

18-20. (canceled)

21. The synthetic GSH of claim 1, wherein the rescue gene sequence encodes a protein.

22. The synthetic GSH of claim 3, wherein the transgene sequence encodes a protein.

23. (canceled)

24. The synthetic GSH of claim 1, wherein the exogenous fusion sequence does not comprise a transgene sequence that encodes a Cas nuclease product, or a gRNA product.

25. The synthetic GSH of claim 1, wherein the synthetic GSH is inserted in a transcriptionally active region of the genome; wherein the synthetic GSH is inserted in a gene cluster region of the genome; and/or wherein the synthetic GSH is inserted in a DNase I hypersensitive site (DHS) of the genome.

26-28. (canceled)

29. A method of making a synthetic GSH in a genome of a cell, the method comprising:
inserting an exogenous fusion sequence at the locus of an endogenous target gene of the genome, wherein the insertion of the fusion sequence inactivates the endogenous target gene, wherein the fusion sequence comprises:
(a) a transgene sequence encoding a transgene product, and
(b) a complementation sequence comprising a rescue gene sequence that encodes a target gene product.

30. The method of claim 29, wherein the insertion of the fusion sequence inactivates only the endogenous target gene and does not inactivate any other genes of the genome; and/or wherein the complementation sequence is capable of rescuing the inactivated endogenous target gene.

31. (canceled)

32. The method of claim 29, comprising delivering a targeted nuclease to the cell.

33-39. (canceled)

40. The method of claim 29, wherein the synthetic GSH is inserted into the genome via homology directed repair (HDR).

41. A method of making a synthetic GSH in a genome of a cell, the method comprising:
inserting a first exogenous fusion sequence at the locus of an endogenous target gene of the genome, wherein the insertion of the fusion sequence inactivates the endogenous target gene, wherein the first fusion sequence comprises:
(a) a landing sequence comprising at least one cutting sequence, and
(b) a complementation sequence comprising a rescue gene sequence that encodes a target gene product.

42. The method of claim 41, wherein the insertion of the fusion sequence inactivates only the endogenous target gene and does not inactivate any other genes of the genome; and/or wherein the complementation sequence is capable of rescuing the inactivated endogenous target gene.

43. The method of claim 41, further comprising inserting a second exogenous fusion sequence into the landing sequence, wherein the second fusion sequence comprises a transgene sequence encoding the transgene product.

44. (canceled)

45. The method of claim 41, comprising delivering a first targeted nuclease to the cell. 46-51. (Canceled)

52. The method of claim 41, wherein the synthetic GSH is inserted into the genome via homology directed repair (HDR).

53-60. (canceled)

61. A method of delivering a gene of interest to a cell comprising a synthetic GSH, the method comprises inserting a sequence comprising a transgene sequence encoding the transgene product into a landing pad of the synthetic GSH, wherein the synthetic GSH is according to claim 2.

62. A synthetic GSH produced by the method of claim 29.

63. (canceled)

64. A cell or a non-human organism comprising the synthetic GSH of claim 1.

65. A polynucleotide or a vector comprising the exogenous fusion sequence according to claim 1.

66-67. (canceled)

68. A synthetic GSH produced by the method of claim 41.
